(19) United States
(12) Patent Application Publication
(10) Pub. No.: US 2010/0143949 A1
(43) Pub. Date: Jun. 10, 2010

(54) BIOMARKERS FOR COLORECTAL CANCER

(75) Inventors: Emanuel Petricoin, Gainesville, VA (US); Weidong Zhou, Manassas, VA (US); Serena Camerini, Napoli (IT); Maria Letizia Polci, Chieti (IT); Lance Liotta, Bethesda, MD (US)

Correspondence Address: FOLEY AND LARDNER LLP;SUITE 500, 3000 K STREET NW, WASHINGTON, DC, 20007, US

(73) Assignees: George Mason Intellectual Properties, Inc.; Istituto Superiore di Sanita

(21) Appl. No.: 12/446,937

(22) PCT Filed: Oct. 31, 2007
(86) PCT No.: PCT/US07/22968

§ 371 Date: Dec. 17, 2009

Related U.S. Application Data
(60) Provisional application No. 60/855,379, filed on Oct. 31, 2006.

(51) Int. Cl. G01N 33/53; G01N 33/00; C12Q 1/37; C07K 16/18

(52) U.S. Cl. 435/7.25; 436/86; 435/7.92; 435/23; 530/387.1; 530/388.1; 530/389.1; 530/387.3

(57) ABSTRACT

Low molecular weight (LMW) peptides have been discovered that are indicative of colorectal cancer. Evaluating patient samples for the presence of such LMW peptides is an effective means of detecting colorectal cancer and monitoring the progression of the disease. The LMW peptides are particularly useful in detecting colorectal cancer during its early stages.

### CROSS-REFERENCE TO RELATED APPLICATIONS

This application claims priority to U.S. Provisional Application No. 60/855,379, filed Oct. 31, 2006, which is hereby incorporated by reference.

### BACKGROUND

Colorectal cancer (CRC) is the second most deadly cancer, causing approximately 500,000 deaths per year. Early detection is paramount to reducing the mortality associated with this disease. Yet, present screening methods are less than effective. Furthermore, present methods are time-consuming, costly and inconvenient for patients.

Colonoscopy is the most commonly used screening method. A variety of factors limit the effectiveness of this method, however. For example, changes in the colon are sometimes invisible during a colonoscopy, and biopsies must be taken during the procedure and examined under a microscope to detect cancerous or precancerous changes. Also, the method's accuracy depends on the experience of the practitioner. Thus, results from different colonoscopies can vary and precancerous changes may go undetected. Colonoscopy also causes patient discomfort and carries certain risks, such as bleeding or puncture of the lining of the colon.

Thus, there is a need for a quick, reliable, non-invasive test for colorectal cancer.

### SUMMARY

In one embodiment, a method for detecting colorectal cancer in a patient comprises obtaining a biological sample from the patient and evaluating the sample or a fraction of the sample for the presence of at least one biomarker selected from the group of peptides having the sequences of SEQ ID NOs: 1-388, wherein the presence of said at least one biomarker is indicative of colorectal cancer. In another, the methods involve evaluating the sample for the presence of a biomarker selected from the group of peptides having the amino acid sequence of SEQ ID NOs: 176-388. In another embodiment, the methods comprise evaluating the sample for the presence of peptides having the amino acid sequence of SEQ ID NOs: 176, 177, and 234.

In one aspect, the colorectal cancer is in early stage, such as stage T1 or T2. The biological sample can be, for example, blood, serum or plasma. In another, the evaluation step comprises assays such as mass spectrometry, an immunoassay such as ELISA, immuno-mass spectrometry or suspension bead array.

In another embodiment, the method further comprises, prior to the evaluation step, harvesting low molecular weight peptides from the biological sample to generate at least one fraction comprising the peptides. In one embodiment, the size of the low molecular weight peptides is less than 50 KDa, preferably less than 25 KDa, and more preferably less than 15 KDa. In another aspect, the method also comprises digesting the low molecular weight peptides. Such digestion can be accomplished using enzymatic or chemical means. In one example, trypsin can be used to digest the peptides.

In another aspect, a method for monitoring the progression of colorectal cancer in a patient comprises (i) obtaining a biological sample from the patient, (ii) evaluating the sample or a fraction of the sample for the presence of at least one biomarker selected from the group of peptides having the sequences of SEQ ID NOs: 1-388, wherein the presence of said at least one biomarker is indicative of colorectal cancer, and optionally, repeating steps (i) and (ii) as necessary. In another, the methods involve evaluating the sample for the presence of a biomarker selected from the group of peptides having the amino acid sequence of SEQ ID NOs: 176-388. In another embodiment, the methods comprise evaluating the sample for the presence of peptides having the amino acid sequence of SEQ ID NOs: 176, 177, and 234. In one embodiment, the method further comprises a step of harvesting low molecular weight peptides from the sample to generate at least one fraction comprising the peptides.

In other aspects, the invention relates to antibodies specific for identified biomarkers for colorectal cancer, as well as kits for detecting colorectal cancer in a patient, comprising at least one such antibody.

Other objects, features and advantages will become apparent from the following detailed description. The detailed description and specific examples are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE DRAWINGS

FIG. 1 provides a CID Spectrum of peptide “TFSLSSTLLR” from Zinc finger protein 553 identified only in LMW of colorectal cancer serum (accession number Q4G0R3, amino acid residues 367-376).

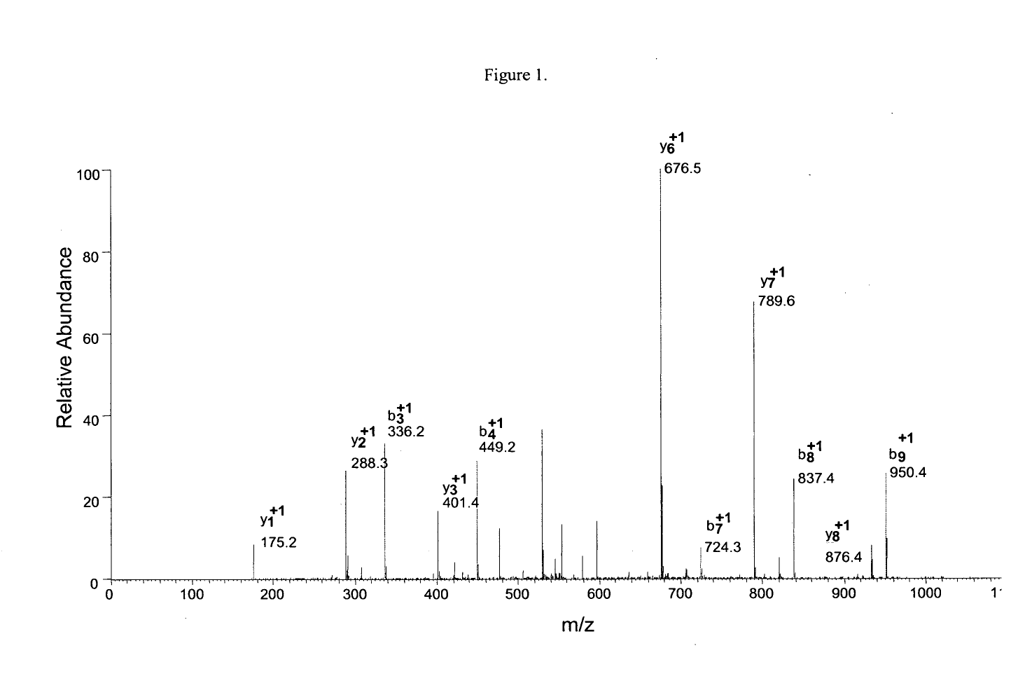

FIG. 2 provides a CID Spectrum of peptide “DMKPENLLCMGPELVK” from Serine/threonine-protein kinase MAK identified only in LMW of colorectal cancer serum (accession number P20794, amino acid residues 125-140).

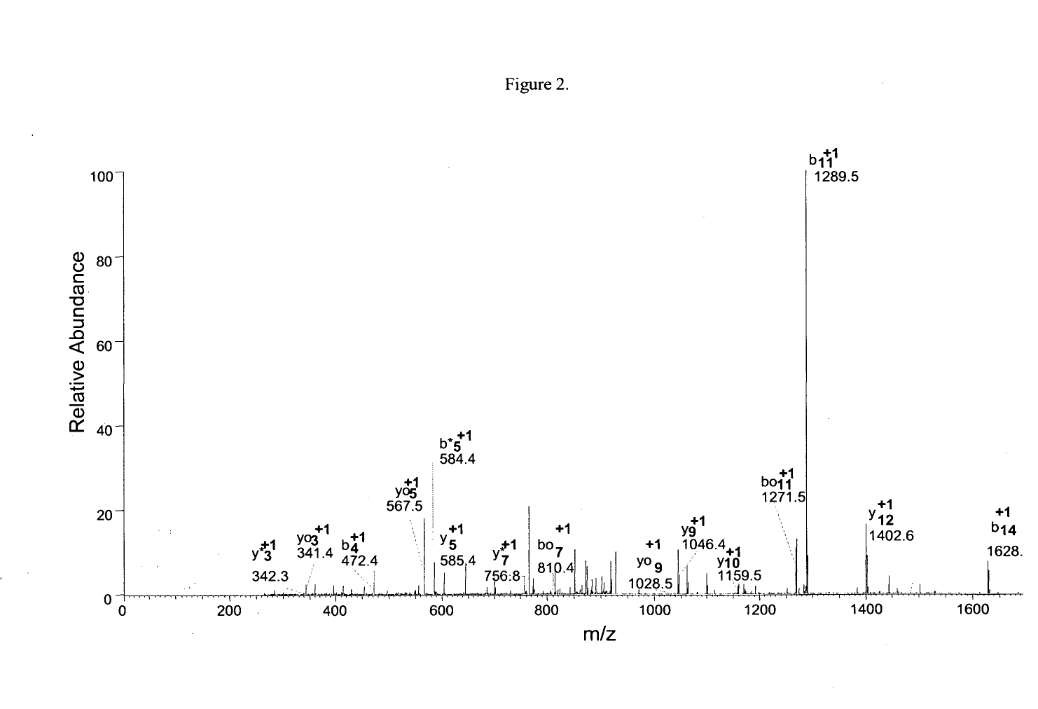

FIG. 3 provides a CID Spectrum of peptide “SGVQQLIQYYQDQK” from Apolipoprotein F precursor identified only in LMW of colorectal cancer serum (accession number Q13790, amino acid residues 233-246).

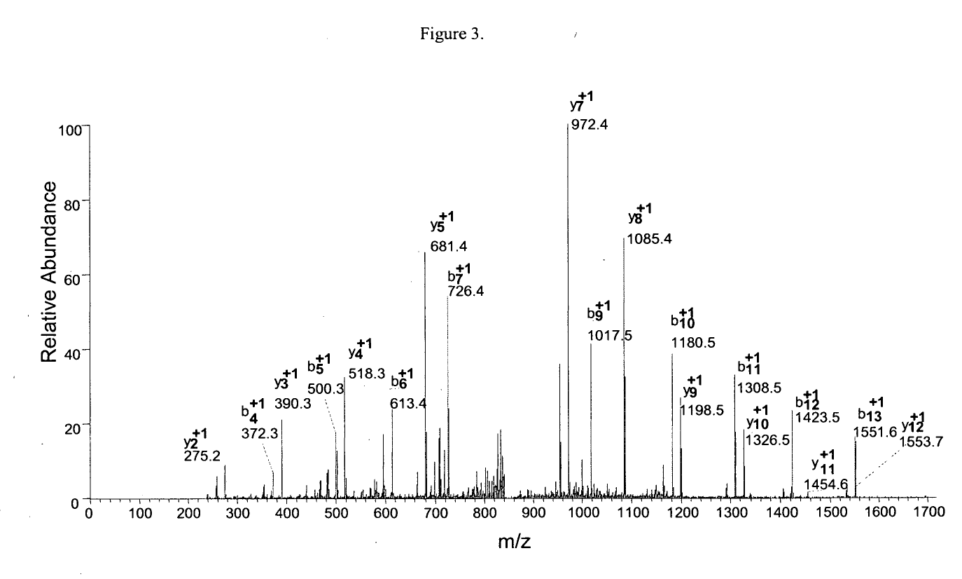

### DETAILED DESCRIPTION

Low molecular weight (LMW) peptides have been discovered that are indicative of colorectal cancer. Evaluating patient samples for the presence of such LMW peptides is an effective means of detecting colorectal cancer and monitoring the progression of the disease, for example during treatment. The LMW peptides are particularly useful in detecting colorectal cancer during its early stages.

The LMW peptides, or biomarkers, can be detected using a variety of methods known in the art. For example, antibodies can be utilized in immunoassays to detect the presence of a biomarker. Exemplary immunoassays include, e.g., ELISA, radioimmunoassay, immunofluorescent assay, “sandwich” immunoassay, western blot, immunoprecipitation assay and immunoelectrophoresis assays. In other examples, beads, microbeads, arrays, microarrays, etc. can be applied in detecting the LMW peptides. Exemplary assays that can be used include suspension bead assays (Schwenk et al., “Determination of binding specificities in highly multiplexed bead-based assays for antibody proteomics,” Mol. Cell Proteomics, 6(1): 125-132 (2007)), antibody microarrays (Borrebaeck et al., “High-throughput proteomics using antibody microarrays: an update,” Expert Rev. Mol. Diagn. 7(5): 673-686 (2007)), aptamer arrays (Walter et al., “High-throughput protein arrays: prospects for molecular diagnostics,” Trends Mol. Med. 8(6): 250-253 (2002)), affybody arrays (Renberg et al., “Affibody molecules in protein capture microarrays: evaluation of multidomain ligands and different detection formats,” J. Proteome Res. 6(1): 171-179 (2007)), and reverse phase arrays (VanMeter et al., “Reverse-phase protein microarrays: application to biomarker discorvery and translational medicine,” Expert Rev. Mol. Diagn. 7(5): 625-633 (2007)). The referenced publications are hereby incorporated by reference.

In another example, the inventive biomarkers can be detected using mass spectrometry (MS). One example of this approach is tandem mass spectrometry (MS/MS), which involves multiple steps of mass selection or analysis, usually separated by some form of fragmentation. Most such assays use electrospray ionization followed by two stages of mass selection: a first stage (MS1) selecting the mass of the intact analyte (parent ion) and, after fragmentation of the parent by collision with gas atoms, a second stage (MS2) selecting a specific fragment of the parent, collectively generating a selected reaction monitoring assay. In one embodiment, collision-induced dissociation is used to generate a set of fragments from a specific peptide ion. The fragmentation process primarily gives rise to cleavage products that break along peptide bonds. Because of the simplicity in fragmentation, the observed fragment masses can be compared to a database of predicted masses for known peptide sequences. A number of different algorithmic approaches have been described to identify peptides and proteins from tandem mass spectrometry (MS/MS) data, including peptide fragment fingerprinting (SEQUEST, MASCOT, OMSSA and X!Tandem), peptide de novo sequencing (PEAKS, LuteFisk and Sherenga) and sequence tag based searching (SPIDER, GutenTAG).

Likewise, multiple reaction monitoring (MRM) can be used to identify the inventive biomarkers in patient samples. This technique applies the MS/MS approach to, for example, tryptic digests of the input sample, followed by selected ion partitioning and sampling using MS to objectify and discreetize the analyte if interest by following the exact m/z ion of the tryptic fragment that represents the analyte. Such an approach can be performed in multiplex so that multiple ions can be measured at once, providing an antibody-free method for analyte measurement. See, e.g. Andersen et al., “Quantitative mass spectrometric multiple reaction monitoring assays for major plasma proteins,” Molecular & Cellular Proteomics, 5.4: 573-588 (2006); Whiteaker et al., “Integrated pipeline for mass spectrometry-based discorvery and confirmation of biomarkers demonstrated in a mouse model of breast cancer,” J. Proteome Res. 6(10): 3962-75 (2007). Both publications are incorporated herein by reference.

In another example, the inventive biomarkers can be detected using nanoflow reverse-phase liquid chromatography-tandem mass spectrometry. See, e.g., Domon B, Aebersold R. “Mass spectrometry and protein analysis.” Science, 312(5771):212-7(2006), which is incorporated herein by reference in its entirety. Using this approach, experimentalists obtain peptide fragments, usually by trypsin digest, and generate mass spectrograms of the fragments, which are then compared to a database, such as SEQUEST, for protein identification.

In another aspect, the inventive biomarkers can be detected using immuno-mass spectrometry. See, e.g., Liotta L et al. “Serum peptidome for cancer detection: spinning biologic trash into diagnostic gold.” J Clin Invest.,116(1):26-30 (2006); Nedelkov, “Mass spectrometry-based immunoassays for the next phase of clinical applications,” Expert Rev. Proteomics, 3(6): 631-640 (2006), which are hereby incorporated by reference. Immuno-mass spectrometry provides a means for rapidly determining the exact size and identity of a peptide biomarker isoform present within a patient sample. When developed as a high throughput diagnostic assay, a drop of patient's blood, serum or plasma can be applied to a high density matrix of microcolumns or microwells filled with a composite substratum containing immobilized polyclonal antibodies, directed against the peptide marker. All isoforms of the peptide that contain the epitope are captured. The captured population of analytes including the analyte fragments are eluted and analyzed directly by a mass spectrometer such as MALDI-TOF MS. The presence of the specific peptide biomarker at its exact mass/charge (m/z) location would be used as a diagnostic test result. The analysis can be performed rapidly by simple software that determines if a series of ion peaks are present at defined m/z locations.

In yet another example, the inventive biomarkers can be detected using standard immunoassay-based approaches whereby fragment specific antibodies are used to measure and record the presence of the diagnostic fragments. See, e.g., Naya et al. “Evaluation of precursor prostate-specific antigen isoform ratios in the detection of prostate cancer.” Urol Oncol. 23(1):16-21 (2005). Moreover, additional well known immunoassays, such as ELISAs (Maeda et al., “Blood tests for asbestos-related mesothelioma,” Oncology 71: 26-31 (2006)), microfluidic ELISAs (Lee et al., “Microfluidic enzyme-linked immunosorbent assay technology,” Adv. Clin. Chem. 42: 255-259 (2006)), nanocantilever immunoassays (Kurosawa et al., “Quartz crystal microbalance immunosensors for environmental monitoring,” Biosens Bioelectron, 22(4): 473-481 (2006)), and plasmon resonance immunoassays (Nedelkov, “Development of surface Plasmon resonance mass spectrometry array platform,” Anal. Chem. 79(15): 5987-5990 (2007)) can be employed. The referenced publications are hereby incorporated by reference.

In a further example, the inventive biomarkers can be detected using electrochemical approaches. See, e.g., Lin et al., “Electrochemical immunosensor for carcinoembryonic antigen based on antigen immobilization in gold nanoparticles modified chitosan membrane,” Anal. Sci. 23(9): 1059-1063 (2007).

In one embodiment, the LMW peptides are harvested from a biological sample prior to the evaluation step. For example, 100 μl of serum can be mixed with 2×SDS-PAGE Laemmli Buffer (containing 200 mM DTT), boiled for 10 minutes, and loaded on Prep Cell (Model 491 Prep Cell, Bio-Rad Laboratories, CA) comprising a 5 cm length 10% acrylamide gel. Electrophoresis is performed under a constant voltage of 250V. Immediately after the bromophenol blue indicator dye is eluted from the system, LMW peptides and proteins migrate out of the gel and are trapped in a dialysis membrane in the elution chamber. These molecules can be eluted at a flow rate of 400 ml/min by a buffer with the same composition of the Tris-Glycine running buffer and collected for 10 minutes in one fraction.

Alternatively, LMW peptides can be harvested using from a sample using a capture-particle that comprises a molecular sieve portion and an analyte binding portion as described in U.S. patent application Ser. No. 11/527,727, filed Sep. 27, 2006, which is hereby incorporated by reference. Briefly, either the molecular sieve portion or the analyte binding portion or both comprise a cross-linked region having modified porosity, or pore dimensions sufficient to exclude high molecular weight molecules.

In another embodiment, the LMW peptides are digested prior to detection, so as to reduce the size of the peptides. Such digestion can be carried out using standard methods well known in the field. Exemplary treatments, include but are not limited to, enzymatic and chemical treatments. Such treatments can yield partial as well as complete digestions. One example of an enzymatic treatment is a trypsin digestion.

The inventive biomarkers are particularly useful in detecting colorectal cancer during its early stages, i.e., prior to metastasis and large tumor volume (e.g. greater than 2 cm).

Antibodies specific for the inventive biomarkers can be produced readily using well known methods in the art. See, e.g. J. Sambrook, E. F. Fritsch and T. Maniatis, Molecular Cloning, a Laboratory Manual, second edition, Cold Spring Harbor Laboratory Press, pp. 18.7-18.18, 1989). For example, the inventive biomarkers can be prepared readily using an automated peptide synthesizer. Next, injection of an immunogen, such as (peptide)ₙ-KLH (n=1-30) in complete Freund's adjuvant, followed by two subsequent injections of the same immunogen suspended in incomplete Freund's adjuvant into immunocompetent animals, is followed three days after an i.v. boost of antigen, by spleen cell harvesting. Harvested spleen cells are then fused with Sp2/0-Ag14 myeloma cells and culture supernatants of the resulting clones analyzed for anti-peptide reactivity using a direct-binding ELISA. Fine specificity of generated antibodies can be detected by using peptide fragments of the original immunogen.

In certain embodiments, one or more antibodies directed to the inventive biomarkers is provided in a kit, for use in a diagnostic method. Such kits also can comprise reagents, instructions and other products for performing the diagnostic method.

### Examples

### Example 1

### Identification of Biomarkers for Colorectal Cancer

Blood Collection and Serum Preparation

Blood samples were drawn from patients before the colonoscopy test under full Institutional Review Board approval and patient's consent. Specimens were collected in red-top Vacutainer Tubes and allowed to clot for 1 hour on ice, followed by centrifugation at 4° C. for 10 minutes at 2000 g. The serum supernatant was divided in aliquots and stored in −80° C. until needed. 10 serum samples with negative outcome were pooled in a single control group. 10 serum samples from patients with a diagnosed T1 or T2 stage colorectal cancer were pooled in a single disease group. Each experiment has been performed using 3 different aliquots from the same pool, both for the control and for the disease group.

Low Molecular Weight (LMW) Protein Harvesting by Continuous Elution Electrophoresis

100 μl of serum was mixed with 2×SDS-PAGE Laemmli Buffer (containing 200 mM DTT), boiled for 10 minutes, and loaded on Prep Cell (Model 491 Prep Cell, Bio-Rad Laboratories, CA) in which 5 cm length 10% acrylamide gel was polymerized. Electrophoresis was performed under a constant voltage of 250V. Immediately after the bromophenol blue indicator dye was eluted from the system, LMW peptides and proteins migrate out of the gel and are trapped in a dialysis membrane in the elution chamber. These molecules were eluted at a flow rate of 400 μl/min by a buffer with the same composition of the Tris-Glycine running buffer and collected for 10 minutes in one fraction.

SOS Removal from the Prep Cell Fractions

LMW fractions obtained by the Prep Cell were processed using a commercially available ion-exchange matrix (Proteo Spin Detergent Clean-Up Micro Kit, Norgen Biotek Corporation, Canada) following protocols outlined by the manufacturer for both acidic and basic proteins, resulting in a final volume of 55 μl.

Nanoflow Reversed-Phase Liquid Chromatography-Tandem MS (nanoRPLC-MS/MS)

The SDS-free LMW fractions obtained from the described procedure were analyzed by traditional bottom-up MS approaches. This was accomplished by treating the samples by reduction using 20 mM DTT, followed by alkylation using 100 mM iodoacetamide and lastly, trypsin digestion (Promega, Wis.) at 37° C. overnight in 50 mM ammonium bicarbonate in the presence of 1M urea in a final volume of 200 μl. Tryptic peptides were desalted by μC₁₈ Zip Tip (Millipore, Mass.) and analyzed by reversed-phase liquid chromatography nanospray tandem mass spectrometry using a linear ion-trap mass spectrometer (LTQ, ThermoElectron, San Jose, Calif.). Reverse phase column was slurry-packed in-house with 5 μm, 200 Å pore size C₁₈ resin (Michrom BioResources, CA) in 100 μm i.d.×10 cm long fused silica capillary (Polymicro Technologies, Phoenix, Ariz.) with a laser-pulled tip. After sample injection, the column was washed for 5 min with mobile phase A (0.4% acetic acid, 0.005% heptafluorobutyric acid) and peptides were eluted using a linear gradient of 0% mobile phase B (0.4% acetic acid, 0.005% heptafluorobutyric acid, 80% acetonitrile) to 50% mobile phase B in 30 min at 250 nl/min, then to 100% B in an additional 5 min. The LTQ mass spectrometer was operated in a data-dependent mode in which each full MS scan was followed by five MS/MS scans where the five most abundant molecular ions were dynamically selected and fragmented by collision-induced dissociation (CID) using a normalized collision energy of 35%.

Bioinformatic Analysis

Tandem mass spectra were matched against Swiss-Prot human protein database through SEQUEST algorithm incorporated in Bioworks software (version 3.2, Thermo Electron) using tryptic cleavage constraints and static cysteine alkylation by iodoacetamide. For a peptide to be considered legitimately identified, it had to achieve Delta Cn value above 0.1, cross correlation scores of 1.5 for [M+H]¹⁺, 2.0 for [M+2H]²⁺, 2.5 for [M+3H]³⁺, and a probability cut-off for randomized identification of p<0.01.

The results are provided in Table 1. In short, 175 peptides were identified as biomarkers that correlate to the disease state. Thus, evaluating patient samples for the presence of one or more of these biomarkers will provide a useful method for detecting colorectal cancer.

TABLE 1

| | | | | | | |
|---|---|---|---|---|---|---|
| | | | | | SEQ ID | Residue |
| Proteins | P (pro) | MW | Accession | Amino acid sequence | NO. | number |

| | | | | | | |
|---|---|---|---|---|---|---|
| SOCS2_HUMAN (O14508) | 8.25E-03 | 22158.33 | O14508 | TGPEAPRNGTVHLYLTKPLYTSAP | 1 | 138-168 |
| Suppressor of cytokine | | | | SLQHLCR | | |
| signaling 2 (SOCS-2) | | | | | | |
| (Cytokine-inducible SH2 protein | | | | | | |
| 2) | | | | | | |
| PGS1_HUMAN (P21810) | 5.33E-03 | 41627.58 | P21810 | VPSGLPDLKLLQVVYLHSNNITK | 2 | 292-314 |
| Biglycan precursor | | | | | | |
| (Bone/cartilage proteoglycan I) | | | | | | |
| (PG-S1) | | | | | | |
| KCC4_HUMAN (Q16566) | 4.36E-03 | 51892.92 | Q16566 | IVEKGYYSERDAADAVK | 3 | 130-146 |
| Calcium/calmodulin-dependent | | | | | | |
| protein kinase type IV (EC | | | | | | |
| 2.7.1.123) (CAM kinase | | | | | | |
| ZF260_HUMAN (Q3ZCT1) Zinc | 9.82E-03 | 46890.02 | Q3ZCT1 | HHTGEKPYECEK | 4 | 101-112 |
| finger protein 260 homolog | | | | | | |
| (Zfp-260) | | | | | | |
| GP107_HUMAN (Q5VW38) | 8.28E-03 | 66948.05 | Q5VW38 | KQSVSVTLLILDISR | 5 | 113-127 |
| Protein GPR107 precursor | | | | | | |
| (Lung seven transmembrane | | | | | | |
| receptor 1) | | | | | | |
| Q6UVY4 (Q6UVY4) | 4.18E-03 | 65025.7 | Q6UVY4 | NYIILNMTENIDCEVFRQHR | 6 | 390-409 |
| Podocalyxin-like protein | | | | | | |
| ELOA2_HUMAN (Q8IYF1) | 2.13E-03 | 83896.48 | Q8IYF1 | YLTYDQLRKQK | 7 | 386-396 |
| RNA polymerase II transcription | | | | | | |
| factor SIll subunit A2 (Elongin | | | | | | |
| A2) (EloA2) (T | | | | | | |
| ZN462_HUMAN (Q96JM2) Zinc | 6.58E-03 | 161426.3 | Q96JM2 | SLLENAEAK | 8 | 1402-1410 |
| finger protein 462 | | | | | | |
| UDB28_HUMAN (Q9BY64) | 2.13E-03 | 60866.27 | Q9BY64 | ENVMKLSIIQHDQPVK | 9 | 440-455 |
| UDP-glucuronosyltransferase | | | | | | |
| 2B28 precursor (EC 2.4.1.17) | | | | | | |
| (UDPGT) | | | | | | |
| A1ATR_HUMAN (P20848) | 7.51E-03 | 47860.97 | P20848 | TEILEGLNVNLTETPEAK | 10 | 100-117 |
| Alpha-1-antitrypsin-related | | | | | | |
| protein precursor | | | | | | |
| Q52LR0 (QS2LR0) Cingulin-like | 4.48E-03 | 149014 | Q52LR0 | RQVEEAEEEIDRLESSK | 11 | 1213-1229 |
| 1 | | | | | | |
| Q5J915 (Q5J915) Migration- | 5.97E-03 | 8760.694 | Q5J915 | RGKPEILELEGLILLK | 12 | 37-52 |
| inducing protein 7 | | | | | | |
| Q8WWY8 (Q8WWY8) | 1.83E-03 | 50826.5 | Q8WWY8 | INHEPTTFQK | 13 | 363-372 |
| Membrane-bound phosphatidic | | | | | | |
| acid-selective phospholipase | | | | | | |
| A1 (Lipase H) (Lipase, mem | | | | | | |
| MUCEN_HUMAN (Q9ULC0) | 1.22E-03 | 27435.32 | Q9ULCO | TISHESGEHSAQGKTK | 14 | 245-260 |
| Endomucin precursor | | | | | | |
| (Endomucin-2) (Gastric cancer | | | | | | |
| antigen Ga34) | | | | | | |
| NALOL_HUMAN (Q9UQQ1) N- | 4.75E-04 | 80574.27 | Q9UQQ1 | YVLYGNHRDSWVHGAVDPSSGTAV | 15 | 362-391 |
| acetylated-alpha-linked acidic | | | | LLELSR | | |
| dipeptidase-like protein (EC | | | | | | |
| 3.4.17.21) (NAAL | | | | | | |
| PEPL_HUMAN (O60437) | 7.80E-04 | 204524.7 | O60437 | QLENEVKSTQEEIWTLR | 16 | 908-924 |
| Periplakin (195 kDa cornified | | | | | | |
| envelope precursor protein) | | | | | | |
| (190 kDa paraneoplast | | | | | | |
| ABLM3_HUMAN (O94929) | 1.26E-03 | 77751.66 | O94929 | EEIKHGQSLLALDK | 17 | 156-169 |
| Actin-binding LIM protein 3 | | | | | | |
| (Actin-binding LIM protein | | | | | | |
| family member 3) (abLIM | | | | | | |
| Q12800 (Q12800) Transcription | 6.35E-03 | 57276.84 | Q12800 | ANPTQLNTVEFLWDPAK | 18 | 165-181 |
| factor LSF | | | | | | |
| Q6PJ08 (Q6PJ08) SLC29A4 | 6.04E-04 | 56365.03 | Q6PJ08 | DSPAHEVTGSGGAYMRFDVPR | 19 | 295-315 |
| protein | | | | | | |
| Q6ZPC4 (Q6ZPC4) | 4.13E-03 | 13386.64 | Q6ZPC4 | NDPLLGMCSEMGFSGVRESFQR | 20 | 102-123 |
| Hypothetical protein FLJ26077 | | | | | | |
| Q8IY84 (Q8IY84) Hypothetical | 9.65E-03 | 49574.63 | Q81Y84 | SILEGTYSVPPHVSEPCHRLIR | 21 | 282-303 |
| protein MGC42105 | | | | | | |
| TR137_HUMAN (O94972) | 1.34E-04 | 107837.5 | O94972 | YEYRVEMVHQSCNDPTKNIIR | 22 | 341-361 |
| Tripartite motif protein 37 | | | | | | |
| (Mulibrey nanism protein) | | | | | | |
| Q495G5 (Q495G5) MMAA | 1.09E-03 | 47105.97 | Q495G5 | VGLSGPPGAGKSTFIEYFGK | 23 | 146-165 |
| protein | | | | | | |
| Q4G0R3 (Q4G0R3) Zinc finger | 8.23E-03 | 67736.06 | Q4GOR3 | TFSLSSTLLR | 24 | 367-376 |
| protein 553 | | | | | | |
| Q659A1 (Q659A1) Hypothetical | 6.70E-03 | 109923.9 | Q659A1 | FMMSKNPSVPVSAVFMDK | 25 | 349-366 |
| protein DKFZp31 312410 | | | | | | |
| Z183L_HUMAN (Q8IZP6) Zinc | 5.85E-03 | 36235.56 | Q8IZP6 | ETGFCGFGDSCKFLHDR | 26 | 201-217 |
| finger protein 183-like 1 (RING | | | | | | |
| finger protein 161) | | | | | | |
| Q8TEE6 (Q8TEE6) FLJ00251 | 2.44E-03 | 110414.6 | Q8TEE6 | AAGQCNNMGQKR | 27 | 754-765 |
| protein (Fragment) | | | | | | |
| PRP18_HUMAN (Q99633) Pre- | 1.63E-03 | 39834.98 | Q99633 | ESITDIIKFMLQR | 28 | 248-260 |
| mRNA splicing factor 18 | | | | | | |
| (PRP18 homolog) (hPRP18) | | | | | | |
| UBP21_HUMAN (Q9UK80) | 9.83E-03 | 62616.42 | Q9UK80 | TSGPRPRGPLR | 29 | 73-83 |
| Ubiquitin carboxyl-terminal | | | | | | |
| hydrolase 21 (EC 3.1.2.15) | | | | | | |
| (Ubiguitin_thiolesteras | | | | | | |
| PSD3_HUMAN (O43242) 26S | 2.65E-03 | 60939.61 | O43242 | EREQQDLEFAKEMAEDDDDSFP | 30 | 513-534 |
| proteasome non-ATPase | | | | | | |
| regulatory subunit 3 (26S | | | | | | |
| proteasome regulatory subun | | | | | | |
| MYG_HUMAN (P02144) | 2.18E-03 | 17041.91 | P02144 | HGATVLTALGGILK | 31 | 64-77 |
| Myoglobin | | | | | | |
| ITPR3 HUMAN (014573) | 9.83E-04 | 303842.3 | Q14573 | NVRSMPLIYWFSR | 32 | 2187-2199 |
| Inositol 1,4,5-trisphosphate | | | | | | |
| receptor type 3 (Type 3 inositol | | | | | | |
| 1,4,5-trisphosph | | | | | | |
| ESPL1_HUMAN (Q14674) | 1.10E-03 | 232963.8 | Q14674 | GVSLLLSVLRDPALQK | 33 | 877-892 |
| Separin (EC 3.4.22.49) | | | | | | |
| (Separase) (Caspase-like | | | | | | |
| protein ESPL1) (Extra spindle | | | | | | |
| Q573B4 (Q573B4) Proto- | 7.31E-03 | 58296.41 | Q573B4 | HYTRYYNGHTK | 34 | 266-276 |
| oncogene tyrosine-protein | | | | | | |
| kinase LCK | | | | | | |
| DAAM2_HUMAN (Q86T65) | 9.87E-03 | 123420.4 | Q86T65 | RGLRAVEVELEYQR | 35 | 886-899 |
| Disheveled associated activator | | | | | | |
| of morphogenesis 2 | | | | | | |
| VCIP1_HUMAN (Q96JH7) | 4.82E-03 | 134235.9 | Q96JH7 | CLLCGALSELHVPPEWLAPGGK | 36 | 469-490 |
| Deubiquitinating protein | | | | | | |
| VC1P135 (EC 3.4.22.-) | | | | | | |
| (Valosin-containing protein p97 | | | | | | |
| THYG_HUMAN (P01266) | 6.86E-03 | 304534.4 | P01266 | RFLAVQSVISGR | 37 | 283-294 |
| Thyroglobulin precursor | | | | | | |
| Q8IXT1 (Q8IXT1) Hypothetical | 7.64E-03 | 111478.8 | Q8IXT1 | ESDHSSLNNK | 38 | 566-575 |
| protein FLJ25416 | | | | | | |
| (Q9BVY9) ZNF160 protein | 8.21E-03 | 17100.86 | Q9BVY9 | TPECVKGVVTDLLR | 39 | 81-94 |
| IC1_HUMAN (P05155) Plasma | 5.14E-03 | 55119.49 | P05155 | FQPTLLTLPR | 40 | 391-400 |
| protease C1 inhibitor precursor | | | | | | |
| (C1 Inh) (C1Inh) | | | | | | |
| KGP1B_HUMAN (P1461 9) | 3.56E-03 | 77754.71 | P14619 | PATQQAQKQSASTLQGEPRTK | 41 | 55-75 |
| cGMP-dependent protein | | | | | | |
| kinase 1, beta isozyme (EC | | | | | | |
| 2.7.1.37) (cGK 1 beta) (cGKI | | | | | | |
| Q5D862 (Q5D862) Ifapsoriasin | 7.33E-03 | 247926.3 | Q5D862 | ASHFQSHSSERQRHGSSQVWK | 42 | 2325-2345 |
| Q6ZQY5 (Q6ZQY5) | 7.81E-03 | 119930.9 | Q6ZQY5 | YENFEDPMGTIDK | 43 | 428-440 |
| Hypothetical protein FLJ46795 | | | | | | |
| RB612_HUMAN (Q8IUD2) | 2.83E-03 | 128007.2 | Q8IUD2 | LLEMLQSKGLSAKATEEDHER | 44 | 312-332 |
| RAB6 interacting protein 2 | | | | | | |
| (ERG protein 1) | | | | | | |
| S13A3_HUMAN (Q8WWT9) | 1.14E-03 | 66797.6 | Q8WWT9 | AQETVPWNIILLLGGGFAMAK | 45 | 421-441 |
| Solute carrier family 13 member | | | | | | |
| 3 (Sodium-dependent high- | | | | | | |
| affinity dicarboxylat | | | | | | |
| UBP29_HUMAN (Q9HBJ7) | 2.80E-03 | 104090.1 | Q9HBJ7 | NMLKEIDKTSFYSICNK | 46 | 124-140 |
| Ubiquitin carboxyl-terminal | | | | | | |
| hydrolase 29 (EC 3.1.2.15) | | | | | | |
| (Ubiguitin thiolesteras | | | | | | |
| VNN3_HUMAN (Q9NY84) | 4.12E-04 | 56054.02 | Q9NY84 | EEALLLMNKNIDVLEK | 47 | 48-63 |
| Vascular non-inflammatory | | | | | | |
| molecule 3 precursor (Vanin-3) | | | | | | |
| MAK_HUMAN (P20794) | 3.64E-03 | 70536.42 | P20794 | DMKPENLLCMGPELVK | 48 | 125-140 |
| Serine/threonine-protein kinase | | | | | | |
| MAK (EC 2.7.1.37) (Male germ | | | | | | |
| cell-associated kin | | | | | | |
| RPB2_HUMAN (P30876) DNA- | 4.42E-03 | 133810.7 | P30876 | NKTQISLVR | 49 | 1142-1150 |
| directed RNA polymerase II 140 | | | | | | |
| kDa polypeptide (EC 2.7.7.6) | | | | | | |
| (RNA polymerase | | | | | | |
| UBE2I_HUMAN (P63279) | 3.13E-03 | 17995.2 | P63279 | LRMLFKDDYPSSPPK | 50 | 60-74 |
| Ubiquitin-conjugating enzyme | | | | | | |
| E2 I (EC 6.3.2.19) (Ubiquitin- | | | | | | |
| protein ligase I) ( | | | | | | |
| PEBB_HUMAN (Q13951) Core- | 3.46E-03 | 21494.7 | Q13951 | EYVDLEREAGKVYLK | 51 | 84-98 |
| binding factor, beta subunit | | | | | | |
| (CBF-beta) (Polyomavirus | | | | | | |
| enhancer binding pro | | | | | | |
| Q5NV64 (Q5NV64) V2-11 | 4.01E-06 | 10532.11 | Q5NV64 | DSERPSGIPER | 52 | 50-60 |
| protein (Fragment) | | | | | | |
| Q6GMV9 (Q6GMV9) | 2.04E-08 | 25629.86 | Q6G MV9 | YLAWYQQKPGQAPR | 53 | 53-66 |
| Hypothetical protein | | | | | | |
| Q7Z2U7 (Q7Z2U7) | 1.41E-03 | 24999.29 | Q7Z2U7 | LTVLRQPK | 54 | 125-132 |
| Hypothetical protein | | | | | | |
| FOG1_HUMAN (Q8IXO7) Zinc | 8.46E-03 | 104481.6 | Q81X07 | PTEEEPGSPWSGPDELEPVVQDGQ | 55 | 77-101 |
| finger protein ZFPM1 (Zinc | | | | R | | |
| finger protein multitype 1) | | | | | | |
| (Friend of GATA pro | | | | | | |
| Q8N4L2 (Q8N4L2) Hypothetical | 3.39E-03 | 28062.26 | Q8N4L2 | FNTLAKCPHCKK | 56 | 168-179 |
| protein TMEM55A | | | | | | |
| Q9H8C8 (Q9H8C8) | 4.38E-03 | 148896.6 | Q9H8C8 | LTEVADFHHAASKVLR | 57 | 188-203 |
| Hypothetical protein FLJ13755 | | | | | | |
| CRP_HUMAN (P02741) C- | 9.64E-06 | 25022.68 | P02741 | KAFVFPK | 58 | 25-31 |
| reactive protein precursor | | | | | | |
| [Contains: C-reactive protein(1- | | | | | | |
| 205)] | | | | | | |
| CRP_HUMAN (P02741) C- | 9.64E-06 | 25022.68 | P02741 | RQDNEILIFWSK | 58 | 76-87 |
| reactive protein precursor | | | | | | |
| [Contains: C-reactive protein(1- | | | | | | |
| 205)] | | | | | | |
| CRP_HUMAN (P02741) C- | 9.64E-06 | 25022.68 | P02741 | YEVQGEVFTKPQLWP | 58 | 210-224 |
| reactive protein precursor | | | | | | |
| [Contains: C-reactive protein(1- | | | | | | |
| 205)] | | | | | | |
| CFAI_HUMAN (P05156) | 3.59E-04 | 65676.66 | P05156 | HGNTDSEGIVEVK | 59 | 118-130 |
| Complement factor I precursor | | | | | | |
| (EC 3.4.21.45) (C3B/C4B | | | | | | |
| inactivator) [Contains: C | | | | | | |
| DPP4_HUMAN (P27487) | 8.28E-03 | 88222.52 | P27487 | RIQNYSVMDICDYDESSGRWNCLV | 60 | 318-343 |
| Dipeptidyl peptidase 4 (EC | | | | AR | | |
| 3.4.14.5) (Dipeptidyl peptidase | | | | | | |
| IV) (DPP IV) (T-cell | | | | | | |
| APOF_HUMAN (Q13790) | 2.03E-06 | 33442.05 | Q13790 | SGVQQLIQYYQDQK | 61 | 233-246 |
| Apolipoprotein F precursor | | | | | | |
| (Apo-F) (Lipid transfer inhibitor | | | | | | |
| protein) (LTIP) | | | | | | |
| PUM1_HUMAN (Q14671) | 6.75E-03 | 126394.8 | Q14671 | MIDVAEPGQR | 62 | 1128-1137 |
| Pumilio homolog 1 (Pumilio-1) | | | | | | |
| (HsPUM) | | | | | | |
| Q5NV67 (Q5NV67) V1-11 | 2.53E-11 | 10481.04 | Q5NV67 | LLIYYDDLLPSGVSDR | 63 | 47-62 |
| protein (Fragment) | | | | | | |
| Q68CZ6 (Q68CZ6) | 9.82E-03 | 69606.88 | Q68CZ6 | WAEESLHSLTSK | 64 | 292-303 |
| Hypothetical protein | | | | | | |
| DKFZp686I1 868 | | | | | | |
| LCTL_HUMAN (Q6UWM7) | 4.68E-03 | 65046.79 | Q6UWM7 | ELHVNHYRFSLSWPR | 65 | 102-116 |
| Lactase-like protein precursor | | | | | | |
| (Klotho/Lactase-phlorizin | | | | | | |
| hydrolase-related prot | | | | | | |
| TECTB_HUMAN (Q96PL2) | 6.77E-03 | 36931.6 | Q96PL2 | EAPFVLEASEIGSDLFAGVEAKGL | 66 | 170-198 |
| Beta-tectorin precursor | | | | SIRFK | | |
| UHRF1_HUMAN (Q96T88) | 5.95E-03 | 89756.99 | Q96T88 | LGLTMQYPEGYLEALANRER | 67 | 601-620 |
| Ubiquitin-like containing PHD | | | | | | |
| and RING finger domains | | | | | | |
| protein 1 (EC 6.3.2.-) ( | | | | | | |
| TRIM7_HUMAN (Q90029) | 7.86E-03 | 56595.02 | Q9C029 | KELEDCEVFRSTEK | 68 | 186-199 |
| Tripartite motif protein 7 | | | | | | |
| (Glycogenin-interacting protein) | | | | | | |
| (RING finger prote | | | | | | |
| RMP_HUMAN (O94763) RNA | 1.85E-03 | 56729.11 | 094763 | KLLPLSVTPEAFSGTVIEK | 69 | 439-457 |
| polymerase II subunit 5- | | | | | | |
| mediating protein (RPB5- | | | | | | |
| mediating protein) | | | | | | |
| Q5NV69 (Q5NV69) V1-13 | 6.37E-09 | 10328.92 | Q5NV69 | LLIYGNSNRPSGVPDR | 70 | 48-63 |
| protein (Fragment) | | | | | | |
| Q68DN6 (Q68DN6) | 5.28E-03 | 196563.1 | Q68DN6 | FDAEISQWKERGLGNLK | 71 | 1054-1070 |
| Hypothetical protein | | | | | | |
| DKFZp781D1923 | | | | | | |
| Q6XYE5 (Q6XYES) FP17548 | 7.90E-03 | 23803.52 | Q6XYE5 | GTLPETPPLPLRSEMR | 72 | 178-193 |
| Q96MC4 (Q96MC4) | 1.89E-036 | 9795.48 | Q96MC4 | AELEKER | 73 | 536-542 |
| Hypothetical protein FLJ32655 | | | | | | |
| SRPK1_HUMAN (Q96SB4) | 5.28E-03 | 92349.12 | Q96SB4 | SAEHYTETALDEIRLLK | 74 | 284-300 |
| Serine/threonine-protein kinase | | | | | | |
| SRPK1 (EC 2.7.1 .37) | | | | | | |
| (Serine/arginine-rich prot | | | | | | |
| Q9H371 (Q9H371) PR01495 | 6.96E-03 | 8858.949 | Q9H371 | SHHPSESLLTVGSILNSLVRLQRS | 75 | 51-74 |
| KINH_HUMAN (P33176) | 2.44E-03 | 109616.9 | P33176 | VHEMEKEHLNK | 76 | 680-690 |
| Kinesin heavy chain | | | | | | |
| (Ubiquitous kinesin heavy | | | | | | |
| chain) (UKHC) | | | | | | |
| UBE3A_HUMAN (Q05086) | 2.30E-03 | 100581.4 | Q05086 | AFRRGFHMVTNESPLK | 77 | 732-747 |
| Ubiquitin-protein ligase E3A | | | | | | |
| (EC 6.3.2.-) (E6AP ubiquitin- | | | | | | |
| protein ligase) (Onc | | | | | | |
| QSNV85 (Q5NV85) V2-8 | 5.35E-07 | 10508.96 | Q5NV85 | ITCGGNNIGSK | 78 | 20-30 |
| protein (Fragment) | | | | | | |
| NOTC4_HUMAN (Q99466) | 2.54E-03 | 209478.2 | Q99466 | APSCGFHHCHHGGLCLPSPK | 79 | 1088-1107 |
| Neurogenic locus notch | | | | | | |
| homolog protein 4 precursor | | | | | | |
| (Notch 4) (hNotch4) [Contai | | | | | | |
| ZNF71_HUMAN (Q9NQZ8) | 8.47E-03 | 54462.41 | Q9NQZ8 | AFSQNMHLIVHQRTHTGEK | 80 | 250-268 |
| Endothelial zinc finger protein | | | | | | |
| induced by tumor necrosis | | | | | | |
| factor alpha (Zinc f | | | | | | |
| Q9UL82 (Q9UL82) Myosin- | 8.88E-05 | 11437.69 | Q9UL82 | DTERPSGIPER | 81 | 50-60 |
| reactive immunoglobulin light | | | | | | |
| chain variable region | | | | | | |
| (Fragment) | | | | | | |
| APOD_HUMAN (P05090) | 2.08E-05 | 21261.77 | P05090 | IPTTFENGR | 82 | 52-60 |
| Apolipoprotein D precursor | | | | | | |
| (Apo-D) (ApoD) | | | | | | |
| APOD HUMAN (P05090) | 2.08E-05 | 21261.77 | P05090 | VLNQELR | 82 | 76-82 |
| Apolipoprotein D precursor | | | | | | |
| (Apo-D) (ApoD) | | | | | | |
| Q5NV81 (Q5NV81) V1-16 | 3.96E-09 | 10277.88 | QSNV81 | LLIYSNNQRPSGVPDR | 83 | 47-62 |
| protein (Fragment) | | | | | | |
| O95267 (O95267) Calcium and | 1.35E-03 | 90272.02 | 095267 | ATQTESQPWIGSEGPSGPFVLSSP | 84 | 672-696 |
| DAG-regulated guanine | | | | R | | |
| nucleotide exchange factor II | | | | | | |
| LV1D_HUMAN (P01702) Ig | 4.33E-04 | 11446.54 | P01702 | LLIYDNNKRPSGIPDR | 85 | 47-62 |
| lambda chain V-I region NIG-64 | | | | | | |
| LV2D_HUMAN (P01707) Ig | 7.97E-06 | 11553.64 | P01707 | SGDTASLTISGLR | 86 | 69-81 |
| lambda chain V-II region TRO | | | | | | |
| HBAZ_HUMAN (P02008) | 2.21E-03 | 15496.2 | P02008 | VDPVNFK | 87 | 93-99 |
| Hemoglobin zeta subunit | | | | | | |
| (Hemoglobin zeta chain) (Zeta- | | | | | | |
| globin) (HBAZ) | | | | | | |
| Q15043 (QQ5043) KIAAOO62 | 1.27E-03 | 58379.58 | Q15043 | LLLPPPAAWDLAVRLR | 88 | 10-25 |
| protein (Fragment) | | | | | | |
| Q5VWOO (Q5VWOO) Novel | 4.91E-03 | 50770.95 | Q5VWOO | FLEERASSSLDSMPGPAGR | 89 | 382-400 |
| protein | | | | | | |
| Q68C18 (Q68C18) Hypothetical | 3.80E-03 | 48598.88 | Q68C18 | ELDIGISATYCGAHSVPK | 90 | 199-216 |
| protein HMFT1272 (Fragment) | | | | | | |
| Q6NS96 (Q6NS96) IGLV3-21 | 5.62E-07 | 24883.22 | Q6NS96 | LSGSNSGNTATLTISR | 91 | 80-95 |
| protein | | | | | | |
| Q6S362 (Q6S362) Usher | 6.05E-03 | 575234.6 | Q6S362 | LSSATPTSLQVVWSTPAR | 92 | 2443-2460 |
| syndrome 2A isoform B | | | | | | |
| (Autosomal recessive, mild) | | | | | | |
| SMS1_HUMAN (Q86VZ5) | 3.25E-04 | 49175 | Q86VZ5 | EMIKIPMPELERSQYPMEWGK | 93 | 120-140 |
| Phosphatidylcholine:ceramide | | | | | | |
| cholinephosphotransferase 1 | | | | | | |
| (EC 2.7.-.-) (Transmem | | | | | | |
| RYR2_HUMAN (Q92736) | 3.02E-03 | 564136.1 | Q92736 | AFLDNAAEDLEK | 94 | 3059-3070 |
| Ryanodine receptor 2 (Cardiac | | | | | | |
| muscle-type ryanodine | | | | | | |
| receptor) (RyR2) (RYR-2) (C | | | | | | |
| ZMY15_HUMAN (Q9H091) | 7.23E-03 | 77264.05 | Q9H091 | ELESLVPR | 95 | 277-284 |
| Zinc finger MYND domain | | | | | | |
| containing protein 15 | | | | | | |
| KV1E_HUMAN (P01597) Ig | 1.17E-03 | 11653.77 | P01597 | YLNWYQQKPGK | 96 | 32-42 |
| kappa chain V-I region DEE | | | | | | |
| IGF1B_HUMAN (P05019) | 7.48E-03 | 21826.96 | P05019 | NAECRGK | 97 | 186-192 |
| Insulin-like growth factor IB | | | | | | |
| precursor (IGF-IB) | | | | | | |
| (Somatomedin C) (Mechano | | | | | | |
| grow | | | | | | |
| SC24C_HUMAN (P53992) | 1.25E-03 | 118239.2 | P53992 | GTEPFVTGVRGQVPPLVTTNFLVK | 98 | 341-364 |
| Protein transport protein | | | | | | |
| Sec24C (SEC24-related protein | | | | | | |
| C) | | | | | | |
| CENB2_HUMAN (Q15057) | 7.20E-03 | 87973.21 | Q15057 | LDKLVKLCIAMIDTGK | 99 | 35-50 |
| Centaurin-beta 2 (Cnt-b2) | | | | | | |
| Q5JZ85 (Q5JZ85) | 4.18E-03 | 24449.01 | Q5JZ85 | SYSCQVMHEGSTAEK | 100 | 202-216 |
| OTTHUMP00000028776 | | | | | | |
| Q5NV79 (Q5NV79) V5-4 | 2.91E-06 | 10672.02 | Q5NV79 | FSGSSSGADR | 101 | 66-75 |
| protein (Fragment) | | | | | | |
| Q5SGD5 (Q5SGD5) Maguin- | 3.65E-03 | 61848.88 | Q5SGD5 | SSSTEPSLLVSWFTR | 102 | 535-549 |
| like protein variant II (Maguin- | | | | | | |
| like protein variant III) (Maguin- | | | | | | |
| like prote | | | | | | |
| Q8N5F4 (Q8N5F4) IGLC1 | 2.23E-04 | 24945.16 | Q8N5F4 | ITCSGDALPK | 103 | 39-48 |
| protein | | | | | | |
| LV1E_HUMAN (P01 703) Ig | 2.75E-04 | 10897.52 | P01703 | VFGGGTK | 104 | 92-98 |
| lambda chain V-I region NEWM | | | | | | |
| C1QA_HUMAN (P02745) | 2.95E-06 | 26000.19 | P02745 | KGHIYQGSEADSVFSGFLIFPSA | 105 | 223-245 |
| Complement C1q | | | | | | |
| subcomponent, A chain | | | | | | |
| precursor | | | | | | |
| QSNV82 (QSNV82) V4-2 | 7.12E-11 | 11135.42 | QSNV82 | IYWYQQKPGSPPQYLLR | 106 | 35-51 |
| protein (Fragment) | | | | | | |
| Q6ZTG8 (Q6ZTG8) | 3.99E-03 | 115755 | Q6ZTG8 | ELCWLLRDER | 107 | 752-761 |
| Hypothetical protein FLJ44670 | | | | | | |
| Q8TBC9 (Q8TBC9) IGLV3-25 | 3.35E-04 | 24851.22 | Q8TBC9 | DNERPSGIPER | 108 | 69-79 |
| protein | | | | | | |
| FA38A_HUMAN (Q92508) | 9.65E-03 | 232890.5 | Q92508 | RPSRSGGR | 109 | 1426-1433 |
| Protein FAM38A | | | | | | |
| Q96TB2 (Q96TB2) Envelope | 5.65E-04 | 19697.06 | Q96TB2 | IVCLPSGIFFVCGTSAYR | 110 | 55 - 72 |
| protein (Fragment) | | | | | | |
| DACT1_HUMAN (Q9NYF0) | 2.50E-03 | 90118.62 | Q9NYF0 | EAWAKPK | 111 | 663-670 |
| Dapper homolog 1 (hDPR1) | | | | | | |
| (Heptacellular carcinoma novel | | | | | | |
| gene 3 protein) | | | | | | |
| Q9UL86 (Q9UL86) Myosin- | 8.61E-04 | 11920.96 | Q9UL86 | LLIYGTSSR | 112 | 47-55 |
| reactive immunoglobulin kappa | | | | | | |
| chain variable region | | | | | | |
| (Fragment) | | | | | | |
| AFF3_HUMAN (P51826) | 1.57E-04 | 133651.8 | P51826 | PKADSQLQPHGGDLTK | 113 | 917-932 |
| AF4/FMR2 family member 3 | | | | | | |
| (LAF-4 protein) (Lymphoid | | | | | | |
| nuclear protein related to A | | | | | | |
| Q5T8Z0 (Q5T8Z0) | 8.09E-04 | 18172.26 | Q5T8Z0 | STLSIGRSLPHITDVSWRLEYQIK | 114 | 81-104 |
| Chromosome 10 open reading | | | | | | |
| frame 8 (Fragment) | | | | | | |
| Q8TE63 (Q8TE63) | 4.70E-05 | 11471.7 | Q8TE63 | LLIYDNNKRPSGVPDR | 115 | 47-62 |
| Immunglobulin light chain | | | | | | |
| variable region (Fragment) | | | | | | |
| Q96LP2 (Q96LP2) Hypothetical | 6.45E-03 | 47696.67 | Q96LP2 | KLSQNIEILEDQIRAR | 116 | 135-150 |
| protein FLJ25333 | | | | | | |
| CK5P1_HUMAN (Q96SZ6) | 7.74E-05 | 67645.74 | Q96SZ6 | ITSASSQTLR | 117 | 576-585 |
| CDK5 regulatory subunit- | | | | | | |
| associated protein 1 (CDK5 | | | | | | |
| activator-binding protein C | | | | | | |
| SNPH_HUMAN (O15079) | 1.28E-03 | 57953.73 | O15079 | RQGQPIYNISSLLR | 118 | 493-506 |
| Syntaphilin | | | | | | |
| UNC5C_HUMAN (O95185) | 2.45E-03 | 103036 | O95185 | RTRTCTNPAPLNGGAFCEGQSVQK | 119 | 283-306 |
| Netrin receptor UNOSO | | | | | | |
| precursor (Unc-5 homolog C) | | | | | | |
| (Unc-5 homolog 3) | | | | | | |
| PRS8_HUMAN (P621 95) 26S | 4.74E-03 | 45597.1 | P62195 | IEFPPPNEEARLDILK | 120 | 315-330 |
| protease regulatory subunit 8 | | | | | | |
| (Proteasome subunit p45) | | | | | | |
| (p45/SUG) (Proteasom | | | | | | |
| OCRL_HUMAN (Q01968) | 5.61E-03 | 104138 | Q01968 | EPCALTLAQRNGQYELIIQLHEK | 121 | 26-48 |
| Inositol polyphosphate 5- | | | | | | |
| phosphatase OCRL-1 (EC | | | | | | |
| 3.1.3.36) (Lowe's oculocerebror | | | | | | |
| M4K2_HUMAN (Q12851) | 1.21E-03 | 91528.26 | Q12851 | TRWTQNFHHFLK | 122 | 239-250 |
| Mitogen-activated protein | | | | | | |
| kinase kinase kinase kinase 2 | | | | | | |
| (EC 2.7.1.37) (MAPKIERK | | | | | | |
| TP53B_HUMAN (Q12888) | 8.07E-03 | 213440.8 | Q12888 | AADISLDNLVEGKRK | 123 | 1614-1628 |
| Tumor suppressor p53-binding | | | | | | |
| protein 1 (p53-binding protein | | | | | | |
| 1) (53BP1) | | | | | | |
| Q5BJF6 (Q5BJF6) ODF | 27.71E-03 | 95341.83 | Q5BJF6 | KIDSLMNAVGCLKSEVK | 124 | 126-142 |
| protein | | | | | | |
| Q6PK04 (Q6PKO4) MGC16597 | 6.85E-03 | 33211.11 | Q6PK04 | NQAIRQPEVQAAPK | 125 | 142-155 |
| protein | | | | | | |
| Q6ZTZ5 (Q6ZTZ5) Hypothetical | 8.36E-03 | 109095.2 | Q6ZTZ5 | TEVEISDHNDSLLMKPLRFR | 126 | 68-87 |
| protein FLJ44101 | | | | | | |
| Q6ZUU4 (Q6ZUU4) | 3.16E-04 | 65663.22 | Q6ZUU4 | KMKATNSLGAGIIEK | 127 | 415-429 |
| Hypothetical protein FLJ43328 | | | | | | |
| TB182_HUMAN (Q9C002) 182 | 2.01E-03 | 181703.8 | Q9COC2 | DEDGSTLFRGWSQEGPVK | 128 | 210-227 |
| kDa tankyrase 1-binding protein | | | | | | |
| GRLF1_HUMAN (Q9NRY4) | 8.92E-03 | 172119.9 | Q9NRY4 | GDNAVIPYETDEDPRRR | 129 | 1200-1216 |
| Glucocorticoid receptor DNA- | | | | | | |
| binding factor 1 (Glucocorticoid | | | | | | |
| receptor repressi | | | | | | |
| U171_HUMAN (Q12980) | 5.46E-03 | 63564.88 | Q12980 | RIATVLQHEER | 130 | 148-158 |
| CGTHBA protein (-14 gene | | | | | | |
| protein) | | | | | | |
| Q8IXL9 (Q8IXL9) 10 motif | 6.96E-03 | 19614.73 | Q8IXL9 | RQAALIAYATR | 131 | 89-99 |
| containing F2 | | | | | | |
| PVR2_HUMAN (Q92692) | 3.34E-03 | 57706.01 | Q92692 | PKNQAEAQK | 132 | 162-170 |
| Poliovirus receptor related | | | | | | |
| protein 2 precursor (Herpes | | | | | | |
| virus entry mediator B) | | | | | | |
| MYO5C_HUMAN (Q9NQX4) | 1.73E-03 | 202665.4 | Q9NQX4 | LYNNFVNRNPLFEK | 133 | 525-538 |
| Myosin-5C (Myosin Vc) | | | | | | |
| Q9UHB7 (Q9UHB7) AF5q31 | 1.25E-04 | 127381.5 | Q9UHB7 | AHLTKLK | 134 | 293-299 |
| protein | | | | | | |
| BAIP2_HUMAN (Q9UQB8) | 6.61E-03 | 60829.68 | Q9UQB8 | SLSPPQSQSKLSDSYSNTLPVR | 135 | 323-344 |
| Brain-specific angiogenesis | | | | | | |
| inhibitor 1-associated protein 2 | | | | | | |
| (BAI1-associated | | | | | | |
| CAD13_HUMAN (P55290) | 1.05E-03 | 78238.09 | P55290 | QAVPDKVWKISK | 136 | 625-636 |
| Cadherin-1 3 precursor | | | | | | |
| (Truncated-cadherin) (T- | | | | | | |
| cadherin) (T-cad) (Heart- | | | | | | |
| cadheri | | | | | | |
| NOG2_HUMAN (Q13823) | 5.25E-03 | 83603.43 | Q13823 | QISVGFIGYPNVGKSSVINTLRSK | 137 | 310-333 |
| Nucleolar GTP-binding protein | | | | | | |
| 2 (Autoantigen NGP-1) | | | | | | |
| Q6ZN84 (06ZN84) | 6.46E-05 | 65950.16 | Q6ZN84 | SFLFDKRPLSPALNALK | 138 | 318-334 |
| Hypothetical protein FLJ16339 | | | | | | |
| Q8N549 (Q8N549) Hypothetical | 1.17E-03 | 27915 | Q8N549 | KFLALQSKNSDADFQNNEK | 139 | 107-125 |
| protein C8orf36 | | | | | | |
| Q8WYY5 (Q8WYY5) | 2.14E-03 | 11844.71 | Q8WYY5 | KMEHHFLATIGLFFWAQGGK | 140 | 18-38 |
| Hypothetical_protein | | | | | | |
| CN133 HUMAN (Q9H9C1) | 1.47E-03 | 56970.36 | Q9H9C1 | VCSLERFRSLQDK | 141 | 169-181 |
| Protein C14orf133 | | | | | | |
| DYH9_HUMAN (Q9NYC9) | 2.61E-03 | 511604.4 | Q9NYC9 | LANPHYQPELQAQATLINFTVTR | 142 | 3557-3579 |
| Ciliary dynein heavy chain 9 | | | | | | |
| (Axonemal beta dynein heavy | | | | | | |
| chain 9) | | | | | | |
| Q9UPW7 (Q9UPW7) KIAA1033 | 5.53E-03 | 138341.3 | Q9UPW7 | FYIFSQFMYDEHIK | 143 | 873-886 |
| protein (Fragment) | | | | | | |
| DYN3_HUMAN (Q9UQ16) | 2.42E-03 | 96621.48 | Q9UQ16 | TGLFTPDMAFEAIVKK | 144 | 400-415 |
| Dynamin-3 (EC 3.6.5.5) | | | | | | |
| (Dynamin, testicular) (T- | | | | | | |
| dynamin) | | | | | | |
| HCFC2_HUMAN (Q9Y5Z7) | 8.55E-03 | 86724.45 | Q9Y5Z7 | ILVFGGMVEYGRYSNELYELQASR | 145 | 84-111 |
| Host cell factor 2 (HCF-2) (C2 | | | | WLWK | | |
| factor) | | | | | | |
| O00319 (O00319) | 7.88E-03 | 74438.16 | O00319 | HFNIHSWEKK | 146 | 512-521 |
| WUGSC:H_DJ525N14.1 | | | | | | |
| protein | | | | | | |
| PHF2_HUMAN (O75151) PHD | 1.57E-03 | 121156.9 | O75151 | KRVLNVTNLEFSDTR | 147 | 187-201 |
| finger protein 2 (GROS) | | | | | | |
| PLST_HUMAN (P13797) T- | 4.92E-04 | 70391.24 | P13797 | AVGDGIVLCKMINLSVPDTIDER | 148 | 156-178 |
| plastin (Plastin-3) | | | | | | |
| Q5KSL6 (Q5KSL6) | 6.79E-03 | 141738.7 | Q5KSL6 | GLYDDTTAFLDEK | 149 | 1154-1166 |
| Diacylglycerol kinase kappa | | | | | | |
| Q6PJZ1 (Q6PJZ1) C9orf58 | 2.36E-03 | 19565.95 | Q6PJZ1 | RLAEINREFLCDQK | 150 | 51-64 |
| protein (Fragment) | | | | | | |
| ARP10_HUMAN (Q9NZ32) | 2.74E-03 | 46277.34 | Q9NZ32 | ANCVAWLGGAIFGALQDILGSRSV | 151 | 350-375 |
| Actin-related protein 10 | | | | SK | | |
| (hARP11) | | | | | | |
| MCE1_HUMAN (O60942) | 6.87E-04 | 68512.69 | O60942 | FHPSMLSNYLKSLKVK | 152 | 45-60 |
| mRNA capping enzyme (HCE) | | | | | | |
| (HCAP1) [Includes: | | | | | | |
| Polynucleotide 5- | | | | | | |
| triphosphatase | | | | | | |
| STXB2_HUMAN (Q15833) | 1.71E-03 | 66396.59 | Q15833 | NGVSEENLAK | 153 | 415-424 |
| Syntaxin binding protein 2 | | | | | | |
| (Unc-18 homolog 2) (Unc-18B) | | | | | | |
| (Unc18-2) | | | | | | |
| KLH17_HUMAN (Q6TDP4) | 7.02E-03 | 69830.2 | Q6TDP4 | QVLELVSSDSLNVPSEEEVYR | 154 | 237-257 |
| Keich-like protein 17 (Actinfilin) | | | | | | |
| ANC4_HUMAN (Q9UJXS) | 7.70E-04 | 91933.59 | Q9UJX5 | YEPLGLDAAGIEEAITAVGSFILK | 155 | 376-399 |
| Anaphase promoting complex | | | | | | |
| subunit 4 (APC4) (Cyclosome | | | | | | |
| subunit 4) | | | | | | |
| SRPX_HUMAN (P78539) Sushi | 6.50E-03 | 51538.23 | P78539 | IQYTVYDRAENK | 156 | 237-248 |
| repeat-containing protein SRPX | | | | | | |
| precursor | | | | | | |
| ZN11B_HUMAN (Q06732) Zinc | 9.02E-04 | 90624.22 | Q06732 | AHTGEKSCQCNECGK | 157 | 714-728 |
| finger protein 11B | | | | | | |
| Q6ZTB6 (Q6ZTB6) | 2.55E-03 | 14350.79 | Q6ZTB6 | RVGHAGFELLTSGDLPASASQSAG | 158 | 75-105 |
| Hypothetical protein FLJ44808 | | | | IAGVSHR | | |
| VASHL_HUMAN (Q86V25) | 2.71E-03 | 40424.05 | Q86V25 | SRSSHARPVSLATSGGSEEEDK | 159 | 22-43 |
| Vasohibin-like protein | | | | | | |
| CKSP2_HUMAN (Q96SN8) | 2.75E-03 | 214903.6 | Q96SN8 | KMHEGDLAMALVLDEK | 160 | 204-219 |
| CDK5 regulatory subunit- | | | | | | |
| associated protein 2 (CDKS | | | | | | |
| activator-binding protein C | | | | | | |
| FBX6_HUMAN (Q9NRD1) F- | 8.94E-03 | 33910.88 | Q9NRD1 | VTNSSIWSPKMTR | 161 | 250-263 |
| box only protein 6 (F-box/G- | | | | | | |
| domain protein 2) | | | | | | |
| MGR5_HUMAN (P41594) | 6.46E-03 | 132383.6 | P41594 | EMGKDYFDYINVGSWDNGELK | 162 | 473-493 |
| Metabotropic glutamate | | | | | | |
| receptor 5 precursor (mGIuR5) | | | | | | |
| AP3S2_HUMAN (P59780) | 5.33E-04 | 22003.34 | P59780 | NINLPEIPRNINIGDLNIKVPNLSQFV | 163 | 167-193 |
| Adapter-related protein | | | | | | |
| complex 3 sigma 2 subunit | | | | | | |
| (Sigma-adaptin 3b) (AP-3 com | | | | | | |
| Q5H9U9 (Q5H9U9) | 9.27E-03 | 152084.4 | Q5H9U9 | VYRAEYINFLENLK | 164 | 1181-1194 |
| Hypothetical protein | | | | | | |
| DKFZp781D1175 | | | | | | |
| Q5VWL1 (Q5VWL1) | 3.08E-04 | 162847.8 | Q5VWL1 | NWEMAYTDTGMIYFIDHNTKTTTW | 165 | 298-325 |
| Membrane-associated | | | | LDPR | | |
| guanylate kinase-related 3 | | | | | | |
| (MAGI-3) | | | | | | |
| Q8WVY7 (Q8WVY7) Ubiquitin- | 4.36E-03 | 36781.22 | Q8WVY7 | KNTIMFDDIGRNFLMNPQNGLK | 166 | 246-267 |
| like domain containing CTD | | | | | | |
| phosphatase 1 (CTD-like | | | | | | |
| phosphatase domain-con | | | | | | |
| LRP1B_HUMAN (Q9NZR2) | 3.60E-03 | 515158.1 | Q9NZR2 | NGVFRVQKFGHGSVEYLALNIDK | 167 | 4135-4157 |
| Low-density lipoprotein | | | | | | |
| receptor-related protein 1B | | | | | | |
| precursor (Low-density lip | | | | | | |
| REV1_HUMAN (Q9UBZ9) DNA | 1.37E-03 | 138161.2 | Q9UBZ9 | DQTKCAASVGIGSNILLAR | 168 | 600-618 |
| repair protein REVi (EC 2.7.7.- | | | | | | |
| )(Rev1-like terminal | | | | | | |
| deoxycytidyl transfera | | | | | | |
| CU005_HUMAN (Q9Y3R5) | 4.39E-03 | 258059.3 | Q9Y3R5 | KNGGEWDVEKVVIDLGGSR | 169 | 725-743 |
| Protein C21orf5 | | | | | | |
| NFIC_HUMAN (P08651) | 3.05E-03 | 55640.11 | P08651 | SPFNSPSPQDSPR | 170 | 333-345 |
| Nuclear factor 1 C-type | | | | | | |
| (Nuclear factor 1/C) (NF1-C) | | | | | | |
| (NFI-C) (NE-I/C) (CCAAT-bo | | | | | | |
| ITA3_HUMAN (P26006) | 4.27E-03 | 118622.2 | P26006 | DKLRPIIISMNYSLPLR | 171 | 563-579 |
| Integrin alpha-3 precursor | | | | | | |
| (Galactoprotein B3) (GAPB3) | | | | | | |
| (VLA-3 alpha chain) (CD4 | | | | | | |
| AP2B1_HUMAN (P63010) | 4.23E-03 | 104486.2 | P63010 | VIAAMTVGKDVSSLFPDVVNCMQT | 172 | 37-67 |
| Adapter-related protein | | | | DNLELKK | | |
| complex 2 beta 1 subunit (Beta- | | | | | | |
| adaptin) (Plasma membra | | | | | | |
| Q6ZSJ3 (Q6ZSJ3) Hypothetical | 1.08E-03 | 18675.65 | Q6ZSJ3 | VLLAFRGRGSGTDR | 173 | 124-137 |
| protein FLJ45478 | | | | | | |
| SEC8_HUMAN (Q96A65) | 7.30E-03 | 110428.6 | Q96A65 | DASVPLIDVTNLPTPR | 174 | 224-239 |
| Exocyst complex component | | | | | | |
| Sec8 | | | | | | |
| PLCB1_HUMAN (Q9NQ66) 1- | 6.38E-03 | 138479.2 | Q9NQ66 | AKQLAALTLEDEEEVK | 175 | 824-839 |
| phosphatidylinositol-4,5- | | | | | | |
| bisphosphate | | | | | | |
| phosphodiesterase beta 1 (EC | | | | | | |
| 3.1 .4.11) | | | | | | |

In addition, the tandem mass spectra were analyzed using more stringent filtering criteria, with a goal of reducing false positives. In particular, the spectra were analyzed using the filtering alorithms of the Scalfold Software (Proteome Software Inc., Portland Oreg.).

The results are provided in Table 2. In short, 74 peptides were identified that correlate to the disease state. Thus, evaluating patient samples for the presence of one or more of these biomarkers will provide a useful method for detecting colorectal cancer.

TABLE 2

| | | | | | Calculated | |
|---|---|---|---|---|---|---|
| | Protein | | | SEQ | Peptide | |
| | accession | Protein | | ID | Mass | Residue |
| Protein name | numbers | MW (Da) | Peptide sequence | NOs | (AMU) | Number |
| C-reactive protein precursor | CRP_HUMAN | 25021 | KAFVFPK | 176 | 836.5 | 25-31 |
| [Contains: C-reactive | | | | | | |
| protein(1-205)] | | | | | | |
| C-reactive protein precursor | CRP_HUMAN | 25021 | RQDNEILIFWSK | 177 | 1548.8 | 76-87 |
| [Contains: C-reactive | | | | | | |
| protein(1-205)] | | | | | | |
| Ig kappa chain V-I region | KV1J_HUMAN | 12751 | ASQSISSWLAWYQQKPGK | 178 | 2065.1 | 47-64 |
| HK102 precursor | | | | | | |
| (Fragment) | | | | | | |
| Apolipoprotein D precursor | APOD_HUMAN | 21258 | IPTTFENGR | 179 | 1034.5 | 52-60 |
| (Apo-D) (ApoD) | | | | | | |
| Alpha-1B-glycoprotein | A1BG_HUMAN | 54254.4 | SWVPHTFESELSDPVELLVAES | 180 | 2471.2 | 474-495 |
| precursor (Alpha-1-B | | | | | | |
| glycoprotein) | | | | | | |
| Alpha-1B-giycoprotein | A1BG_HUMAN | 54254.4 | TPGAAANLELIFVGPQHAGNYR | 181 | 2296.2 | 448-469 |
| precursor (Alpha-1-B | | | | | | |
| glycoprotein) | | | | | | |
| Hypothetical protein | Q6ZNX5 | 17656.3 | RPSGVSDR | 182 | 873.45 | 73-80 |
| FLJ26936 | | | | | | |
| Ig lambda chain V-IV region | LV4C_HUMAN | 11498.7 | SYELTQPPSVSVSPGQTAR | 183 | 2004 | 1-19 |
| Hil | | | | | | |
| Carbohydrate | CHST8_HUMAN | 48817.7 | EPFERLVSAFRDK | 184 | 1593.8 | 259-271 |
| sulfotransferase 8 (EC | | | | | | |
| 2.8.2.-) (N- | | | | | | |
| acetylgalactosamine-4-O- | | | | | | |
| sulfotransferase 1) | | | | | | |
| (GalNAc-4-O- | | | | | | |
| sulfotransferase 1) | | | | | | |
| (GalNAc-4-ST1) | | | | | | |
| (GalNAc4ST-1) | | | | | | |
| Hypothetical protein | Q8NEJ1 | 25005.7 | NDQRPSGVPDR | 185 | 1240.6 | 71-81 |
| Amyloid lambda 6 light | Q96JD0 | 12276.6 | LTVLGQPK | 186 | 855.53 | 109-116 |
| chain variable region SAR | | | | | | |
| (Fragment) | | | | | | |
| C18orf34 protein | Q5BJE1 | 100177 | YESEIKYLTIMKLK | 187 | 1759 | 477-490 |
| Hypothetical protein | Q7Z2U7 | 24996.3 | LTVLRQPK | 188 | 954.61 | 125-132 |
| FIGNL1 protein | Q6PIW4 | 74092.8 | YHQPQRASGSSYGGVK | 189 | 1721.8 | 310-325 |
| (AMU) | | | | | | |
| Serum amyloid A protein | SAA_HUMAN | 13514.5 | SEESELGEAFOGAR | 190 | 1550.7 | 20-33 |
| precursor (SAA) [Contains: | | | | | | |
| Amyloid protein A (Amyloid | | | | | | |
| fibril protein AA); Serum | | | | | | |
| amyloid protein A(2-104); | | | | | | |
| Serum amyloid protein A(3- | | | | | | |
| 104); Serum amyloid | | | | | | |
| protein A(2-103); Serum | | | | | | |
| amyloid protein A(2-102); | | | | | | |
| Serum amyloid protein A(4- | | | | | | |
| 101 | | | | | | |
| SERPINC1 protein | Q8TCE1 | 29075.3 | ANRPFLVFIR | 191 | 1232.7 | 231-240 |
| Serum amyloid P- | SAMP_HUMAN | 25369.7 | AYSLFSYNTQGR | 192 | 1406.7 | 65-76 |
| component precursor (SAP) | | | | | | |
| (9.5S alpha-1-glycoprotein) | | | | | | |
| [Contains: Serum amyloid | | | | | | |
| P-component(1-203)] | | | | | | |
| Immunoglobulin lambda-like | IGLL1_HUMAN | 22944.9 | YAASSYLSLTPEQWR | 193 | 1771.9 | 173-187 |
| polypeptide 1 precursor | | | | | | |
| (Immunoglobulin-related | | | | | | |
| 14.1 protein) | | | | | | |
| (Immunoglobulin omega | | | | | | |
| polypeptide) (Lambda 5) | | | | | | |
| (CD179b antigen) | | | | | | |
| Hemoglobin gamma-1 | HBG1_HUMAN | 15991.3 | VNVEDAGGETLGR | 194 | 1316.6 | 18-30 |
| subunit (Hemoglobin | | | | | | |
| gamma-i chain) (Gamma- | | | | | | |
| 1-globin) (Hemoglobin | | | | | | |
| gamma-A chain) (Hb F | | | | | | |
| Agamma) | | | | | | |
| Hypothetical protein | Q8N313 | 81587.4 | QIKDETLQAAVR | 195 | 1371.8 | 414-425 |
| DKFZp761 P18121 | | | | | | |
| Zinc finger protein 40 | ZEP1_HUMAN | 297199 | QFTKQNGETPGIIAEASK | 196 | 1919 | 107-124 |
| (Human immunodeficiency | | | | | | |
| virus type I enhancer- | | | | | | |
| binding protein 1) (HIV- | | | | | | |
| EP1) (Major | | | | | | |
| histocompatibility complex | | | | | | |
| binding protein 1) (MBP-1) | | | | | | |
| (Positive regulatory domain | | | | | | |
| II binding factor 1) (PRD!I- | | | | | | |
| BF1) | | | | | | |
| Kininogen-1 precursor | KNG1_HUMAN | 71927.5 | AATGECTATVGK | 197 | 1165.6 | 102-113 |
| (Alpha-2-thiol proteinase | | | | | | |
| inhibitor) [Contains: | | | | | | |
| Kininogen-1 heavy chain; | | | | | | |
| Bradykinin (Kallidin I); | | | | | | |
| Lysyl-bradykinin (Kallidin II); | | | | | | |
| Kininogen-1 light chain; Low | | | | | | |
| molecular weight growth | | | | | | |
| promoting factor] | | | | | | |
| Kininogen-1 precursor | KNG1_HUMAN | 71927.5 | TVGSDTFYSFK | 198 | 1251.6 | 65-75 |
| (Alpha-2-thiol proteinase | | | | | | |
| inhibitor) [Contains: | | | | | | |
| Kininogen-1 heavy chain; | | | | | | |
| Bradykinin (Kallidin I); | | | | | | |
| Lysyl-bradykinin (Kallidin II); | | | | | | |
| Kininogen-1 light chain; Low | | | | | | |
| molecular weight growth | | | | | | |
| promoting factor] | | | | | | |
| Kininogen-1 precursor | KNG1_HUMAN | 71927.5 | YNSQNQSNNQFVLYR | 199 | 1874.9 | 44-58 |
| (Alpha-2-thiol proteinase | | | | | | |
| inhibitor) [Contains: | | | | | | |
| Kininogen-1 heavy chain; | | | | | | |
| Bradykinin (Kallidin I); | | | | | | |
| Lysyl-bradykinin (Kallidin II); | | | | | | |
| Kininogen-1 light chain; Low | | | | | | |
| molecular weight growth | | | | | | |
| promoting factor] | | | | | | |
| Ig kappa chain V-II region | KV2A_HUMAN | 12658.6 | FSGSGSGTDFTLK | 200 | 1303.6 | 69-81 |
| Cum | | | | | | |
| Ig kappa chain V-Il region | KV2A_HUMAN | 12658.6 | FSGSGSGTDFTLKISR | 201 | 1659.8 | 69-84 |
| Cum | | | | | | |
| Netrin receptor UNC5C | UNC5C_HUMAN | 103085 | VYEMYVTVHR | 202 | 1296.6 | 564-573 |
| precursor (Unc-5 homolog | | | | | | |
| C) (Unc-5 homolog 3) | | | | | | |
| VS-4 protein (Fragment) | Q5NV79 | 10660.8 | FSGSSSGADR | 203 | 970.42 | 66-75 |
| Hypothetical protein | Q9NVY1 | 45979 | WGMLFLVNQLFKIYIK | 204 | 2013.1 | 166-181 |
| FLJ10440 | | | | | | |
| Hypothetical protein | Q6N095 | 52341.3 | VTVSSASTK | 205 | 879.48 | 141-149 |
| DKFZp686K03196 | | | | | | |
| Ig lambda chain V-II region | LV2D_HUMAN | 11542.9 | SGDTASLTISGLR | 206 | 1277.7 | 69-81 |
| TRO | | | | | | |
| CCDC28B protein (Novel | Q8TBV8 | 22018.9 | RSPKPCLAQPAQAPGTLR | 207 | 1948.1 | 8-25 |
| protein) | | | | | | |
| Oxysterol binding protein- | OSR2_HUMAN | 55184.3 | FWGKSVEAEPRGTITLELLK | 208 | 2274.3 | 206-225 |
| related protein 2 (OSBP- | | | | | | |
| related protein 2) (ORP-2) | | | | | | |
| Apolipoprotein M (Apo-M) | APOM_HUMAN | 21235.9 | AFLLTPR | 209 | 817.49 | 172-178 |
| (ApoM) (G3a protein) | | | | | | |
| Apolipoprotein M (Apo-M) | APOM_HUMAN | 21235.9 | KWIYHLTEGSTDLR | 210 | 1718.9 | 99-112 |
| (ApoM) (G3a protein) | | | | | | |
| V1-16 protein (Fragment) | Q5NV81 | 10266.2 | LLIYSNNQRPSGVPDR | 211 | 1829 | 47-62 |
| Ig kappa chain V-I region | KV1A_HUMAN | 11974.8 | TFGQGTK | 212 | 738.38 | 97-103 |
| AG | | | | | | |
| Hypothetical protein | Q6GMV9 | 25628.4 | YLAWYQQKPGQAPR | 213 | 1705.9 | 53-66 |
| Apolipoprotein F precursor | APOF_HUMAN | 33445.8 | SGVQQLIQYYQDQK | 214 | 1697.9 | 233-246 |
| (Apo-F) (Lipid transfer | | | | | | |
| inhibitor protein) (LTIP) | | | | | | |
| Hypothetical protein | Q5U5X8 | 46774 | AAVSSSSTAAPAGPAKSVLKSAEGK | 215 | 2272.2 | 108-132 |
| FLJ14721 | | | | | | |
| V2-8 protein (Fragment) | Q5NV85 | 10497.3 | ITCGGNNIGSK | 216 | 1120.5 | 20-30 |
| Protocadherin gamma C3 | PCDGK_HUMAN | 101061 | ETGEMFVNDRLDR | 217 | 1581.7 | 82-94 |
| precursor (PCDH-gamma- | | | | | | |
| C3) (Protocadherin 43) (PC | | | | | | |
| 43)(Protocadherin 2) | | | | | | |
| Protein C21orf5 | CUOO5_HUMAN | 258213 | KNGGEWDVEKVVIDLGGSR | 218 | 2058.1 | 725-743 |
| V2-11 protein (Fragment) | QSNV64 | 10520.9 | DSERPSGIPER | 219 | 1242.6 | 50-60 |
| Microtubule-actin | MACF1_HUMAN | 620397 | ALIAEHQTFMEEMTRKQPDVDR | 220 | 2645.3 | 4960-4981 |
| crosslinking factor 1, | | | | | | |
| isoforms 1/2/3/5 (Actin | | | | | | |
| cross-linking family protein | | | | | | |
| 7)(Macrophin-1) | | | | | | |
| (Trabeculin-alpha) (620 kDa | | | | | | |
| actin-binding protein) | | | | | | |
| (ABP620) | | | | | | |
| Neuronal acetylcholine | ACHAS_HUMAN | 53037.9 | ALRLLLLVQLVAGRCGLAGAAGGAQR | 221 | 2604.5 | 10-35 |
| receptor protein, alpha-5 | | | | | | |
| subunit precursor | | | | | | |
| FLJ35834 protein | Q49AL6 | 92565.1 | HKVTGVEYHNAGTQTVPK | 222 | 1966 | 258-275 |
| OTTHUMP00000028561 | Q5THZ5 | 196693 | PEVAAKPALPTQKPAGTLPR | 223 | 2042.2 | 176-195 |
| V4-2 protein (Fragment) | Q5NV82 | 11124.7 | IYWYQQKPGSPPQYLLR | 224 | 2137.1 | 35-51 |
| PHD finger protein 2 | PHF2_HUMAN | 121218 | KRVLNVTNLEFSDTR | 225 | 1792 | 187-201 |
| (GRC5) | | | | | | |
| Macrophage-stimulating | RON_HUMAN | 152208 | HIDPFDLTHFLAQGRR | 226 | 1923 | 1290-1305 |
| protein receptor precursor | | | | | | |
| (EC 2.7.1.112) (MSP | | | | | | |
| receptor) (p185-Ron) | | | | | | |
| (CDW136) (CD136 antigen) | | | | | | |
| [Contains: Macrophage- | | | | | | |
| stimulating protein receptor | | | | | | |
| alpha chain; Macrophage- | | | | | | |
| stimulating protein receptor | | | | | | |
| beta chain] | | | | | | |
| IBR domain containing | IBRD1_HUMAN | 31963.7 | TRNQTQHLAPQPVLLSDMLYCLK | 227 | 2726.4 | 241-263 |
| protein 1 | | | | | | |
| Hypothetical protein | Q8N825 | 66152.4 | PGPCWPGPSSHANGDPVAVAK | 228 | 2101 | 564-584 |
| FLJ40112 | | | | | | |
| Methylenetetrahydrofolate | Q5JYA3 | 12407.6 | AVIELLEKSGVNLDGKK | 229 | 1813 | 96-112 |
| dehydrogenase (NADP + | | | | | | |
| dependent) 1-like | | | | | | |
| (Fragment) | | | | | | |
| NOD16 | Q7RTR4 | 119414 | TMLPEATLLIMIR | 230 | 1501.8 | 323-335 |
| MGC10701 protein | Q9BSY8 | 5306.9 | MAVTGAGSGAR | 231 | 977.48 | 1-11 |
| Zinc finger protein 462 | ZN462_HUMAN | 161512 | SLLENAEAK | 232 | 974.52 | 1402-1410 |
| C14orf125 protein | Q6PSR9 | 12885.8 | QKTLVEQLLSLLNSSPGPPTRK | 233 | 2406.4 | 49-70 |
| IGLV3-21 protein | Q6N596 | 24879.8 | LSGSNSGNTATLTISR | 234 | 1578.8 | 80-95 |
| Hypothetical protein | Q6AHZ1 | 166734 | ITSVFSLQSQQASEFLPPEVNQLLQD | 235 | 3258.7 | 764-792 |
| DKFZp78102147 | | | VLK | | | |
| Hypotheticalprotein | Q9NUY8 | 76459.1 | VISFIENTSTPVDRHVSSSDRVGK | 236 | 2630.4 | 505-528 |
| FLJ11046 | | | | | | |
| Zinc finger MYND domain | ZMY15_HUMAN | 77295.3 | ELESLVPR | 237 | 942.53 | 277-284 |
| containing protein 15 | | | | | | |
| Band 4.1-like protein 2 | E41L2_HUMAN | 112570 | LVSPEQPPKAK | 238 | 1193.7 | 497-507 |
| (Generally expressed | | | | | | |
| protein 4.1) (4.1G) | | | | | | |
| IGLC1 protein | Q8NSF4 | 24942.4 | ITCSGDALPK | 239 | 1061.5 | 39-48 |
| Transcription factor BTF3 | BT3L1_HUMAN | 12518.3 | QAEVHTGR | 240 | 897.45 | 14-21 |
| homolog 1 (Basic | | | | | | |
| transcription factor 3-like 1) | | | | | | |
| Complement factor I | CFAI_HUMAN | 65701.8 | HGNTDSEGIVEVK | 241 | 1384.7 | 118-130 |
| precursor (EC 3.4.21.45) | | | | | | |
| (C3B/C4B inactivator) | | | | | | |
| [Contains: Complement | | | | | | |
| factor I heavy chain; | | | | | | |
| Complement factor I light | | | | | | |
| chain] | | | | | | |
| V1-11 protein (Fragment) | Q5NV67 | 10469.7 | LLIYYDDLLPSGVSDR | 242 | 1839 | 47-62 |
| Splice variant of four and a | Q5TM15 | 15872.1 | NNMPCSAFSAKSPSPR | 243 | 1750.8 | 98-113 |
| half LIM domain protein 2 | | | | | | |
| RNA polymerase II subunit | RMP_HUMAN | 56746.4 | KLLPLSVTPEAFSGTVIEK | 244 | 2029.2 | 439-457 |
| 5-mediating protein (RPB5- | | | | | | |
| mediating protein) | | | | | | |
| T-complex protein 1, theta | TCPQ_HUMAN | 59471.8 | LYAVHQEGNKNVGLDIEAEVPAVK | 245 | 2593.4 | 466-489 |
| subunit (TCP-1 -theta) | | | | | | |
| (CCT-theta) | | | | | | |
| Seven transmembrane helix | Q8NH06 | 33619.8 | EDTAAAVMYTVVTPLLNPFIYSMR | 246 | 2702.4 | 258-281 |
| receptor | | | | | | |
| Lysozyme C precursor (EC | LYSC_HUMAN | 16518.9 | STDYGIFQINSR | 247 | 1400.7 | 69-80 |
| 3.2.1.17) (1,4-beta-N- | | | | | | |
| acetylmuramidase C) | | | | | | |
| WAP four-disulfide core | Q5JYQ5 | 17950.6 | RSLNNLQDLAHNPGLSPR | 248 | 2002.1 | 141-158 |
| domain 6 | | | | | | |
| PML-RARA regulated | Q8N6W7 | 73980.3 | EKDPQPQQLPPMDPKLLK | 249 | 2102.1 | 547-564 |
| adaptor molecule 1 | | | | | | |

### Example 2

### Identification of Biomarkers for Colorectal Cancer Using LTQ-Orbitrap

Collection of blood and harvest of LMW protein were performed as described in Example 1.

Sample Preparation for Mass Spectrometric Analysis

LMW proteins collected from PrepCell were concentrated by Centricon (Millipore), loaded to SDS-PAGE (4-20% Tris-Glycine, Invitrogen) and proteins were separated by electrophoresis. After Coomassie staining and destaining of the gel, each lane was sliced to 5 bands. Then in-gel digestion by trypsin was performed for each band and peptides were extracted from the gel for mass spectrometric analysis.

Nanoflow Reversed-Phase Liquid Chromatography Tandem Mass Spectrometry

The peptides from each band were analyzed by reversed-phase liquid chromatography nanospray tandem mass spectrometry using LTQ-Orbitrap mass spectrometer (ThermoFisher). Reverse phase column was slurry-packed in-house with 5 μm, 200 Å pore size C₁₈ resin (Michrom BioResources, CA) in 100 μm i.d.×10 cm long fused silica capillary (Polymicro Technologies, Phoenix, Ariz.) with a laser-pulled tip. After sample injection, the column was washed for 5 min with mobile phase A (0.1% formic acid) and peptides were eluted using a linear gradient of 0% mobile phase B (0.1% formic acid, 80% acetonitrile) to 50% mobile phase B in 90 min at 200 nl/min, then to 100% B in an additional 5 min. The LTQ-Orbitrap mass spectrometer was operated in a data-dependent mode in which each full MS scan was followed by five MS/MS scans where the five most abundant molecular ions were dynamically selected and fragmented by collision-induced dissociation (CID) using a normalized collision energy of 35%.

Bioinformatic Analysis

Tandem mass spectra were matched against human database downloaded from the National Center for Biotechnology Information (NCBI) through the Sequest Bioworks Browser (ThermoFisher) using full tryptic cleavage constraints and static cysteine alkylation by iodoacetamide. For a peptide to be considered legitimately identified, it had to be the top number one matched and had to achieve cross correlation scores of 1.9 for [M+H]¹⁺, 2.2 for [M+2H]²⁺, 3.5 for [M+3H]³⁺, ΔCn>0.1, and a maximum probabilities of identification of 0.01.

The results are provided in Table 3. In summary, 139 peptides were identified that correlate to the disease state.

Subsequently, the candidate biomarkers are verified and validated for colorectal cancer, followed by analysis of LMW protein fractions less than 25 KDa and less than 15 KDa from colorectal cancer pooled sera by reverse phase protein array.

LMW protein fractions from individual patient samples with and without colorectal cancer were isolated and collected using continuous denaturing electrphoresis and spotted on a nitrocellulose substratum using a reverse phase array format whereby the LMW sample is diluted 1:1 with SDS sample buffer and printed. The slide is then blocked with casein hydrolysate and incubated with an rabbit polyclonal anti-CRP antibody for 16 hours. The slide is washed and incubated with a horseradish peroxidae coupled goat anti-rabbit and subject to tyrmaide amplification using a colorimetric (DAB) precipitant.

Thus, evaluating patient samples for the presence of one or more of these biomarkers will provide a useful method for detecting colorectal cancer.

TABLE 3

| | | | | | Calculated | |
|---|---|---|---|---|---|---|
| | Protein | | | SEQ | Peptide | |
| | accession | Protein | | ID | Mass | Residue |
| Protein name | numbers | MW (Da) | Peptide sequence | NOs | (AMU) | Number |
| serum amyloid A1 | gi\|40316912\|ref\|NP_000322.2\| | 13514.5 | FFGHGAEDSLADQAANEWGR | 250 | 2178 | 86-105 |
| isoform 1 [Homo | | | | | | |
| sapiens] | | | | | | |
| serum amyloid A1 | gi\|4O316912\|ref\|NP_000322.2\| | 13514.5 | GPGGAWAAEVISDAR | 251 | 1456.7 | 66-80 |
| isoform 1 [Homo | | | | | | |
| sapiens] | | | | | | |
| serum amyloid A1 | gi\|40316912\|ref\|NP_000322.2\| | 13514.5 | RGPGGAWAAEVISDAR | 252 | 1612.8 | 65-80 |
| isoform 1 [Homo | | | | | | |
| sapiens] | | | | | | |
| serum amyloid A1 | gi\|40316912\|ref\|NP_000322.2\| | 13514.5 | SFFSFLGEAFDGAR | 253 | 1550.7 | 20-33 |
| isoform 1 [Homo | | | | | | |
| sapiens] | | | | | | |
| A-gamma globing | gi\|28302131\|ref\|NP_000550.2\| | 16110.4 | LLVVYPWTQR | 254 | 1274.7 | 32-41 |
| [Homo sapiens] | | | | | | |
| delta globin [Homo | gi\|4504351\|ref\|NP_000510.1\| | 16037.1 | WAGVANALAHK | 255 | 1149.7 | 134-145 |
| sapiens] | | | | | | |
| serum amyloid A4, | gi\|10835095\|ref\|NP_006503.1\| | 14789.3 | VYLQGLIDYYLFGNSSTVLED | 256 | 2624.3 | 81-103 |
| constitutive [Homo | | | SK | | | |
| sapiens] | | | | | | |
| apolipoprotein C-III | gi\|4557323\|ref\|NP_000031.1\| | 10834.3 | DALSSVQESQVAQQAR | 257 | 1716.9 | 45-60 |
| precursor [Homo | | | | | | |
| sapiens] | | | | | | |
| apolipoprotein C-III | gi\|4557323\|ref\|NP_000031.1\| | 10834.3 | GWVTDGFSSLK | 258 | 1196.6 | 61-71 |
| precursor [Homo | | | | | | |
| sapiens] | | | | | | |
| platelet factor 4 | gi\|4505733\|ref\|NP_002610.1\| | 10827.5 | AGPHCPTAQLIATLK | 259 | 1577.8 | 63-77 |
| (chemokine (C-X- | | | | | | |
| C motif) ligand 4) | | | | | | |
| [Homo sapiens] | | | | | | |
| platelet factor 4 | gi\|4505733\|ref\|NP_002610.1\| | 10827.5 | HITSLEVIK | 260 | 1039.6 | 54-62 |
| (chemokine (C-X- | | | | | | |
| C motif) ligand 4) | | | | | | |
| [Homo sapiens] | | | | | | |
| platelet factor 4 | gi\|4505733\|ref\|NP_002610.1\| | 10827.5 | ICLDLQAPLYK | 261 | 1333.7 | 82-92 |
| (chemokine (C-X- | | | | | | |
| C motif) ligand 4) | | | | | | |
| [Homo sapiens] | | | | | | |
| platelet factor 4 | gi\|4505733\|ref\|NP_002610.1\| | 10827.5 | KICLDLQAPLYK | 262 | 1461.8 | 81-92 |
| (chemokine (C-X- | | | | | | |
| C motif) ligand 4) | | | | | | |
| [Homo sapiens] | | | | | | |
| apolipoprotein C- | gi\|4502161\|ref\|NP_001637.1\| | 14535.5 | AWFLESK | 263 | 880.46 | 99-105 |
| IV [Homo sapiens] | | | | | | |
| apolipoprotein C- | gi\|4502161\|ref\|NP_001637.1\| | 14535.5 | DGWQWFWSPSTFR | 264 | 1699.8 | 67-79 |
| IV [Homo sapiens] | | | | | | |
| apolipoprotein C- | gi\|4502161\|ref\|NP_001637.1\| | 14535.5 | ELLETWNR | 265 | 1072.6 | 56-64 |
| IV [Homo sapiens] | | | | | | |
| apolipoprotein C- | gi\|4502161\|ref\|NP_001637.1\| | 14535.5 | GFMQTYYDDHLR | 266 | 1545.7 | 80-91 |
| IV [Homo sapiens] | | | | | | |
| proteoglycan 4 | gi\|67190163\|ref\|NP_005798.2\| | 151062.3 | DQYYNIDVPSR | 267 | 1369.6 | 1373-1383 |
| [Homo sapiens] | | | | | | |
| proteoglycan 4 | gi\|67190163\|ref\|NP_005798.2\| | 151062.3 | GLPNVVTSAISLPNIR | 268 | 1651 | 1345-1360 |
| [Homo sapiens] | | | | | | |
| apolipoprotein E | gi\|4557325\|ref\|NP_000032.1\| | 36135.5 | SWFEPLVEDMQR | 269 | 1536.7 | 281-292 |
| precursor [Homo | | | | | | |
| sapiens] | | | | | | |
| alpha-2-HS- | gi\|4502005\|ref\|NP_001613.1\| | 39305.4 | HTFMGWSLGSPSGEVSHPR | 270 | 2081 | 318-337 |
| glycoprotein | | | | | | |
| [Homo sapiens] | | | | | | |
| serpin peptidase | gi\|50659080\|ref\|NP_001076.2\| | 47635 | AVLDVFEEGTEASAATAVK | 271 | 1908 | 361-379 |
| inhibitor, clade | | | | | | |
| A, member 3 | | | | | | |
| precursor [Homo | | | | | | |
| sapiens] | | | | | | |
| serpin peptidase | gi\|50659080\|ref\|NP_001076.2\| | 47635 | FNRPFLMIIVPTDTQNIFFMS | 272 | 2659.4 | 395-416 |
| inhibitor, clade | | | K | | | |
| A, member 3 | | | | | | |
| precursor [Homo | | | | | | |
| sapiens] | | | | | | |
| serpin peptidase | gi\|50659080\|ref\|NP_001076.2\| | 47635 | ITLLSALVETR | 273 | 1215.7 | 380-390 |
| inhibitor, clade | | | | | | |
| A, member 3 | | | | | | |
| precursor [Homo | | | | | | |
| sapiens] | | | | | | |
| complement | gi\|56786155\|ref\|NP_758957.2\| | 25756 | FNAVLTNPQGDYDTSTGK | 274 | 1927.9 | 140-157 |
| component 1, q | | | | | | |
| subcomponent, | | | | | | |
| gamma | | | | | | |
| polypeptide | | | | | | |
| [Homo sapiens] | | | | | | |
| coagulation factor | gi\|4503625\|ref\|NP_000495.1\| | 54714.1 | ACIPTGPYPCGK | 275 | 1320.6 | 163-174 |
| X preproprotein | | | | | | |
| [Homo sapiens] | | | | | | |
| coagulation factor | gi\|4503625\|ref\|NP_000495.1\| | 54714.1 | NCELFTR | 276 | 939.44 | 120-126 |
| X preproprotein | | | | | | |
| [Homo sapiens] | | | | | | |
| complement | gi\|50345296\|ref\|NP_00100202 | 192734.8 | AEMADQAAAWLTR | 277 | 1433.7 | 1279-1291 |
| component 4B | 9.1\| | | | | | |
| preproprotein | | | | | | |
| [Homo sapiens] | | | | | | |
| proline rich 11 | gi\|88703045\|ref\|NP_060774.2\| | 40068.3 | DTIFPSR | 278 | 835.43 | 100-106 |
| [Homo sapiens] | | | | | | |
| angiotensinogen | gi\|4557287\|ref\|NP_000020.1\| | 53136.8 | ALQDQLVLVAAK | 279 | 1268.8 | 83-94 |
| preproprotein | | | | | | |
| [Homo sapiens] | | | | | | |
| complement factor | gi\|11321587\|ref\|NP_002104.1\| | 37643.3 | EIMENYNIALR | 280 | 1365.7 | 271-281 |
| H-related 1 [Homo | | | | | | |
| sapiens] | | | | | | |
| ribonuclease, | gi\|4506557\|ref\|NP_002928.1\| | 16822.4 | ENTEIHEDIWNIR | 281 | 1704.8 | 70-82 |
| RNase A family, 4 | | | | | | |
| precursor [Homo | | | | | | |
| sapiens] | | | | | | |
| ribonuclease, | gi\|4506557\|ref\|NP_002928.1\| | 16822.4 | YCNLMMQR | 282 | 1115.5 | 52-59 |
| RNase A family, 4 | | | | | | |
| precursor [Homo | | | | | | |
| sapiens] | | | | | | |
| serine. (or | gi\|39725934\|ref\|NP_002606.3\| | 46296.3 | DTDTGALLFIGK | 283 | 1250.7 | 400-411 |
| cysteine) | | | | | | |
| proteinase | | | | | | |
| inhibitor, clade F | | | | | | |
| (alpha-2 | | | | | | |
| antiplasmin, | | | | | | |
| pigment epithelium | | | | | | |
| derived factor), | | | | | | |
| member 1 [Homo | | | | | | |
| sapiens] | | | | | | |
| plasminogen- | gi\|4505883\|ref\|NP_002656.1\| | 10952.8 | AFQYHSK | 284 | 880.43 | 63-69 |
| related protein B2 | | | | | | |
| [Homo sapiens] | | | | | | |
| plasminogen- | gi\|4505883\|ref\|NP_002656.1\| | 10952.8 | KSSIIIR | 285 | 816.53 | 81-87 |
| related protein B2 | | | | | | |
| [Homo sapiens] | | | | | | |
| chemokine (C-C | gi\|14589959\|ref\|NP_116738.1\| | 12279.6 | PGIVFITK | 286 | 874.54 | 78-85 |
| motif) ligand 14 | | | | | | |
| isoform 2 | | | | | | |
| precursor [Homo | | | | | | |
| sapiens] | | | | | | |
| sperm | gi\|8394343\|ref\|NP_059121.1\| | 17388.5 | FYNNHAFEEQEPPEK | 287 | 1878.8 | 68-82 |
| autoantigenic | | | | | | |
| protein 17 [Homo | | | | | | |
| sapiens] | | | | | | |
| myosin light chain | gi\|47132569\|ref\|NP_444257.2\| | 161732.9 | FRKQIQESEHMK | 288 | 1560.8 | 1274-1285 |
| kinase isoform 4 | | | | | | |
| [Homo sapiens] | | | | | | |
| UDP-glucose | gi\|9910280\|ref\|NP_064505.1\| | 177176.9 | EPVYLSGYGVELAIK | 289 | 1637.9 | 237-251 |
| ceramide | | | | | | |
| glucosyltransfer- | | | | | | |
| ase- like 1 | | | | | | |
| isoform 1 | | | | | | |
| [Homo sapiens] | | | | | | |
| hypothetical | gi\|34787409\|ref\|NP_065069.1\| | 166783.6 | EPANSR | 290 | 673.33 | 1125-1130 |
| protein LOC57148 | | | | | | |
| [Homo sapiens] | | | | | | |
| serine (or | gi\|50363219\|ref\|NP_00100223 | 46719.9 | FNKPFVFLMIEQNTK | 291 | 1856 | 390-404 |
| cysteine) | 6.1\| | | | | | |
| proteinase | | | | | | |
| inhibitor, clade A | | | | | | |
| (alpha-a | | | | | | |
| antiproteinase | | | | | | |
| antitrypsin), | | | | | | |
| member 1 [Homo | | | | | | |
| sapiens] | | | | | | |
| serine (or | gi\|50363219\|ref\|NP_00100223 | 46719.9 | GTEAAGAMFLEAIPMSIPPEV | 292 | 2259.1 | 368-389 |
| cysteine) | 6.1\| | | | K | | |
| proteinase | | | | | | |
| inhibitor, clade A | | | | | | |
| (alpha-1 | | | | | | |
| antiproteinase, | | | | | | |
| antitrypsin), | | | | | | |
| member 1 [Homo | | | | | | |
| sapiens] | | | | | | |
| serine (or | gi\|50363219\|ref\|NP_00100223 | 46719.9 | LSITGTYDLK | 293 | 1110.6 | 315-324 |
| cysteine) | 6.1\| | | | | | |
| proteinase | | | | | | |
| inhibitor, clade A | | | | | | |
| (alpha-1 | | | | | | |
| antiproteinase, | | | | | | |
| antitrypsin), | | | | | | |
| member 1 [Homo | | | | | | |
| sapiens] | | | | | | |
| serine (or | gi\|50363219\|re\|INP_00100223 | 46719.9 | SPLFMGK | 294 | 779.41 | 405-411 |
| cysteine) | 6.1\| | | | | | |
| proteinase | | | | | | |
| inhibitor, clade A | | | | | | |
| (alpha-1 | | | | | | |
| antiproteinase, | | | | | | |
| antitrypsin), | | | | | | |
| member 1 [Homo | | | | | | |
| sapiens] | | | | | | |
| serine (or | gi\|50363219\|ref\|NP_00100223 | 46719.9 | SVLGQLGITK | 295 | 1015.6 | 325-334 |
| cysteine) | 6.1\| | | | | | |
| proteinase | | | | | | |
| inhibitor, clade A | | | | | | |
| (alpha-1 | | | | | | |
| antiproteinase, | | | | | | |
| antitrypsin), | | | | | | |
| member 1 [Homo | | | | | | |
| sapiens] | | | | | | |
| serine (or | gi\|50363219\|ref\|NP_00100223 | 46719.9 | VFSNGADLSGVTEEAPLK | 296 | 1833.9 | 335-352 |
| cysteine) | 6.1\| | | | | | |
| proteinase | | | | | | |
| inhibitor, clade A | | | | | | |
| (alpha-1 | | | | | | |
| antiproteinase, | | | | | | |
| antitrypsin), | | | | | | |
| member 1 [Homo | | | | | | |
| sapiens] | | | | | | |
| secretoglobin, | gi\|50363226\|ref\|NP_443095.2\| | 10082.3 | LLLSSLGIPVNHLIEGSQK | 297 | 2018.2 | 59-77 |
| family 3A, member | | | | | | |
| 1 [Homo sapiens] | | | | | | |
| fibroblast | gi\|169335401ref1NP_004451.2\| | 87697.2 | TOQEIKILEENKELENALK | 298 | 2257.2 | 481-499 |
| activation | | | | | | |
| protein, | | | | | | |
| alpha subunit | | | | | | |
| [Homo sapiens] | | | | | | |
| PREDICTED: | gi\|88979556\|ref\|XP_945982.\| | 159016.4 | WNHCFLCKKCVK | 299 | 1679.8 | 408-419 |
| similar to Zinc | | | | | | |
| finger CCHC | | | | | | |
| domain-containing | | | | | | |
| protein 4 isoform | | | | | | |
| 5 [Homo sapiens] | | | | | | |
| lipopolysaccha- | gi\|31652249\|ref\|NP_004130.2\| | 53368 | LAEGFPLPLLK | 300 | 1197.7 | 444-454 |
| ride- binding | | | | | | |
| protein | | | | | | |
| precursor [Homo | | | | | | |
| sapiens] | | | | | | |
| sex hormone- | gi\|7382460\|ref\|NP_001031.2\| | 43762.6 | IALGGLLFPASNLR | 301 | 1441.9 | 170-183 |
| binding globulin | | | | | | |
| [Homo sapiens] | | | | | | |
| platelet factor 4 | gi\|4505735\|ref\|NP_002611.1\| | 11535.1 | AEAEEDGDLQCLCVK | 302 | 1736.7 | 34-48 |
| variant 1 [Homo | | | | | | |
| sapiens] | | | | | | |
| peptidyl arginine | gi\|6912576\|ref\|NP_036519.1\| | 74078 | AEAFFPNMVNMLVLGK | 303 | 1812.9 | 579-594 |
| deiminase, type IV | | | | | | |
| [Homo sapiens] | | | | | | |
| discs large | gi\|51339031\|ref\|NP_00100380 | 75244.3 | SLESSQRQEAR | 304 | 1290.6 | 630-640 |
| homolog- | 9.1\| | | | | | |
| associated protein | | | | | | |
| 1 isoform beta | | | | | | |
| [Homo sapiens] | | | | | | |
| caspase 14 | gi\|6912286\|ref\|NP_036246.1\| | 27661.8 | GSCFIQTLVDVFTK | 305 | 1614.8 | 184-197 |
| precursor [Homo | | | | | | |
| sapiens] | | | | | | |
| alpha-2-plasmin | gi\|11386143\|ref\|NP_000925.1\| | 54578.6 | LGNQEPGGQTALK | 306 | 1312.7 | 52-64 |
| inhibitor [Homo | | | | | | |
| sapiens] | | | | | | |
| prolactin-induced | gi\|4505821\|ref\|NP_002643.1\| | 16555.1 | FYTIEILKVE | 307 | 1254.7 | 137-146 |
| protein [Homo | | | | | | |
| sapiens] | | | | | | |
| PREDICTED: | gi\|113420925\|ref\|XP_0011309 | 34923.4 | ACEGAEVREAAR | 308 | 1318.6 | 196-207 |
| hypothetical 79.11 | | | | | | |
| protein [Homo | | | | | | |
| sapiens] | | | | | | |
| pericentriolar | gi\|34878902\|ref\|NP_006188.2\| | 228284.4 | LPEMEPLVPRVKEVK | 309 | 1780 | 1906-1920 |
| material 1 [Homo | | | | | | |
| sapiens] | | | | | | |
| CIpX caseinolytic | gi\|7242140\|ref\|NP_006651.2\| | 69206.7 | SASEGSSKKSGSGNSGK | 310 | 1554.7 | 82-98 |
| protease X | | | | | | |
| homolog [Homo | | | | | | |
| sapiens] | | | | | | |
| leucine rich repeat | gi\|42544231\|ref\|NP_006329.2\| | 78842.3 | RYRVYPEGTLELR | 311 | 1651.9 | 473-485 |
| neuronal 5 | | | | | | |
| precursor [Homo | | | | | | |
| sapiens] | | | | | | |
| 5-azacytidine | gi\|57863291\|ref\|NP_00100981 | 117291.9 | CSELKGQLGEAEGENLR | 312 | 1889.9 | 912-928 |
| induced 1 soform | 1.1\| | | | | | |
| b [Homo sapiens] | | | | | | |
| profilin family, | gi\|40786418\|ref\|NP_955378.1\| | 14301.7 | CVRADEYSLYAKNENTGVVVV | 313 | 2514.3 | 73-94 |
| member 4 [Homo | | | K | | | |
| sapiens] | | | | | | |
| PREDICTED: | gi\|113427383\|ref\|XP_0011262 | 27008.5 | HMKIKSLEEMYVFSLPMK | 314 | 2259.1 | 12-29 |
| similarto | 32.1\| | | | | | |
| ribosomal protein | | | | | | |
| S2 [Homo sapiens] | | | | | | |
| small inducible | gi\|4506831\|ref\|NP_002979.1\| | 9830.9 | FIVDYSETSPQCPK | 315 | 1670.8 | 43-56 |
| cytokine A18 | | | | | | |
| precursor [Homo | | | | | | |
| sapiens] | | | | | | |
| chemokine (C-X-C | gi\|76563933\|ref\|NP_00102905 | 13687.7 | ILNTPNCALQIVAR | 316 | 1582.9 | 49-62 |
| motif) ligand 12 | 8.1\| | | | | | |
| (stromal cell- | | | | | | |
| derived factor 1) | | | | | | |
| isoform gamma | | | | | | |
| (Homo sapiens] | | | | | | |
| heparanase | gi\|94721347\|ref\|NP_006656.21 | 61134.1 | TDFLIFDPK | 317 | 1095.6 | 99-107 |
| [Homo sapiens] | | | | | | |
| keratin associated | gi\|38490587\|ref\|NP_941972.1\| | 25086 | PSSSVSLLCRPVCRPAR | 318 | 1942 | 191-207 |
| protein 10-12 | | | | | | |
| [Homo sapiens] | | | | | | |
| retinitis | gi\|5454016\|ref\|NP_006260.1\| | 240645.3 | KVQPVDLDKAR | 319 | 1268.7 | 111-121 |
| pigmentosa RP1 | | | | | | |
| protein [Homo | | | | | | |
| sapiens] | | | | | | |
| zinc finger protein | gi\|41281441\|ref\|NP_055514.21 | 200730.2 | ENSRTETTMSPPR | 320 | 1521.7 | 603-615 |
| 646 [Homo | | | | | | |
| sapiens] | | | | | | |
| PREDICTED: | gi\|113429344\|ref\|XP_372900.4\| | 13053.9 | SHSAHFFEFLTK | 321 | 1450.7 | 76-87 |
| similar to D- | | | | | | |
| dopachrome | | | | | | |
| decarboxylase (D- | | | | | | |
| dopachrome | | | | | | |
| tautomerase) | | | | | | |
| (Phenylpyruvate | | | | | | |
| tautomerase II) | | | | | | |
| [Homo sapiens] | | | | | | |
| fibulin 1 isoform | gi\|34734066\|ref\|NP_006477.2\| | 77221.7 | AITPPHPASQANIIFDITEGN | 322 | 2475.3 | 620-642 |
| D [Homo sapiens] | | | LR | | | |
| macrophage | gi\|4505185\|ref\|NP_002406.1\| | 12458.5 | LLCGLLAER | 323 | 1044.6 | 79-87 |
| migration inhibitory | | | | | | |
| factor | | | | | | |
| (glycosylation- | | | | | | |
| inhibiting factor) | | | | | | |
| [Homo sapiens] | | | | | | |
| thioredoxin [Homo | gi\|50592994\|ref\|NP_003320.2\| | 11719.3 | TAFQEALDAAGDK | 324 | 1336.6 | 9-21 |
| sapiens] | | | | | | |
| PREDICTED: | gi\|89037862\|ref\|XP_945963.1\| | 155161.2 | QCGLHDGLQDTSQDK | 325 | 1701.8 | 1246-1260 |
| similar to | | | | | | |
| retrotransposon- | | | | | | |
| like 1 [Homo | | | | | | |
| sapiens] | | | | | | |
| \|uiescin Q6 | gi\|13325075\|ref\|NP_002817.2\| | 82560.7 | SALYSPSDPLTLLQADTVR | 326 | 2047.1 | 33-51 |
| isoform a [Homo | | | | | | |
| sapiens] | | | | | | |
| pancreas-enriched | gi\|19923455\|ref\|NP_057425.2\| | 258827.4 | VHFEDLVFLR | 327 | 1274.7 | 1928-1937 |
| phospholipase C | | | | | | |
| [Homo sapiens] | | | | | | |
| leader-binding | gi\|38045903\|ref\|NP_937825.1\| | 49166 | SVIMWFAEDK | 328 | 1253.6 | 113-123 |
| protein 32 isoform | | | | | | |
| 2 [Homo sapiens] | | | | | | |
| transducer of | gi\|56118245\|ref\|NP_073606.2\| | 66969.4 | EENLLNVPKPLPK | 329 | 1490.9 | 214-226 |
| regulated CREB | | | | | | |
| protein 3 [Homo | | | | | | |
| sapiens] | | | | | | |
| G-gammaglobin | gi\|6715607\|ref\|NP_000175.1\| | 16108.5 | KVLTSLGDAIK | 330 | 1144.7 | 67-77 |
| [Homo sapiens] | | | | | | |
| golgi autoantigen, | gi\|4758454\|ref\|NP_004478.1\| | 376057.9 | EISNLNQLIEEFK | 331 | 1576.8 | 717-729 |
| golgin subfamily b, | | | | | | |
| macrogolgin (with | | | | | | |
| transmembrane | | | | | | |
| signal), 1 [Homo | | | | | | |
| sapiens] | | | | | | |
| PREDICTED: | gi\|89031353\|ref\|XP_934040.1\| | 25721.4 | MNQLILTTGGRINTFHGTKK | 332 | 2230.2 | 1-20 |
| similar to | | | | | | |
| eukaryotic | | | | | | |
| translation | | | | | | |
| initiation factor 5A | | | | | | |
| [Homo sapiens] | | | | | | |
| microtubule- | gi\|10346135\|ref\|NP_055083.1\| | 37013.6 | FQDNLDFIQWFK | 333 | 1600.8 | 144-155 |
| associated protein, | | | | | | |
| RP/EB family, | | | | | | |
| member 2 [Homo | | | | | | |
| sapiens] | | | | | | |
| PREDICTED: | gi\|113422814\|ref\|XP_0011325 | 166806.4 | HLGLYESSMMPPGRPR | 334 | 1827.9 | 118-133 |
| hypothetical | 91.1\| | | | | | |
| protein [Homo | | | | | | |
| sapiens] | | | | | | |
| chromosome 9 | gi\|\|11641247\|ref\|NP_071738.1\| | 17200.2 | ASASDGSSFVVAR | 335 | 1253.6 | 120-132 |
| open reading | | | | | | |
| framal9 [Homo | | | | | | |
| sapiens] | | | | | | |
| pancreatic | gi\|38201682\|ref\|NP_937875.1\| | 17625.8 | SNSSMHITDCR | 336 | 1307.5 | 103-113 |
| ribonuclease | | | | | | |
| precursor [Homo | | | | | | |
| sapiens] | | | | | | |
| ADP-ribosylation | gi\|4502209\|ref\|NP_001653.1\| | 20512.1 | DAVLLVFANK | 337 | 1089.6 | 118-127 |
| factor 5 [Homo | | | | | | |
| sapiens] | | | | | | |
| protein S (alpha) | gi\|4506117\|ref\|NP_000304.1\| | 75055.3 | SFQTGLFTAAR | 338 | 1198.6 | 91-101 |
| [Homo sapiens] | | | | | | |
| fibronectin 1 | gi\|47132551\|ref\|NP_997640.1\| | 266182.2 | TYHVGEQWQK | 339 | 1275.6 | 2336-2345 |
| isoform 2 | | | | | | |
| preproprotein | | | | | | |
| [Homo sapiens] | | | | | | |
| myoglobin [Homo | gi\|4885477\|ref\|NP_005359.1\| | 17166.4 | HGATVLTALGGILK | 340 | 1350.8 | 65-78 |
| sapiens] | | | | | | |
| hypothetical | gi\|56711320\|ref\|NP_00100187 | 84154.6 | LGDNEETQVR | 341 | 1160.6 | 330-339 |
| protein | 2.2\| | | | | | |
| LOC145407 | | | | | | |
| [Homo sapiens] | | | | | | |
| G protein-coupled | gi\|89257346\|ref\|NP_005284.2\| | 38631.1 | PGLLHQGRQR | 342 | 1161.7 | 226-235 |
| receptor 20 [Homo | | | | | | |
| sapiens] | | | | | | |
| lysosomal acid | gi\|4557010\|ref\|NP_001601.1\| | 48326.8 | YEQLQNETR | 343 | 1180.6 | 162-170 |
| phosphatase 2 | | | | | | |
| precursor [Homo | | | | | | |
| sapiens] | | | | | | |
| keratin 24 [Homo | gi\|9506669\|ref\|NP_061889.1\| | 55128.6 | \|QSVEADINGLR | 344 | 1201.6 | 242-252 |
| sapiens] | | | | | | |
| neuroblastoma- | gi\|41393547\|ref\|NP_056993.2\| | 268559 | FRMTQLTVEK | 345 | 1268.7 | 832-841 |
| amplified protein | | | | | | |
| [Homo sapiens] | | | | | | |
| PREDICTED: | gi\|89036937\|ref\|XP_944641.1\| | 19303.4 | EGEMEVAMQK | 346 | 1183.5 | 62-71 |
| similar to CDC42- | | | | | | |
| binding protein | | | | | | |
| kinase beta [Homo | | | | | | |
| sapiens] | | | | | | |
| ubi\|uinol- | gi\|46593007\|ref\|NP_003356.2\| | 52627.9 | MAASWCRAATAGAQVLLR | 347 | 1945 | 1-19 |
| cytochrome c | | | | | | |
| reductase core | | | | | | |
| protein I [Homo | | | | | | |
| sapiens] | | | | | | |
| syntaxin 8 [Homo | gi\|4759188\|ref\|NP_004844.1\| | 26889.6 | QNLLDDLVTR | 348 | 1186.6 | 80-89 |
| sapiens] | | | | | | |
| phospholipase D1, | gi\|4505873\|ref\|NP_002653.1\| | 124170.4 | IAADMSNIIENLDTR | 349 | 1691.8 | 17-31 |
| phophatidylcholine- | | | | | | |
| specific [Homo | | | | | | |
| sapiens] | | | | | | |
| titin isoform N2-B | gi\|20143914\|ref\|NP_003310.2\| | 2992949 | LSWKMPDDDGGDRIK | 350 | 1748.8 | 9433-9447 |
| (Homo sapiens] | | | | | | |
| hypothetical | gi\|8923251\|ref\|NP_060208.1\| | 125270 | TAELSVKEYKEVNEK | 351 | 1766.9 | 683-697 |
| protein LOC54875 | | | | | | |
| [Homo sapiens] | | | | | | |
| guanine nucleotide | gi\|38788319\|ref\|NP_005266.2\| | 68642.7 | DSREEVER | 352 | 1019.5 | 88-95 |
| binding protein-like | | | | | | |
| 1 [Homo sapiens] | | | | | | |
| hypothetical | gi\|75832029\|ref\|NP_065880.1\| | 150366.6 | SQSADPFLNLEMDAGISNIOR | 353 | 2306.1 | 1070-1090 |
| protein LOC57589 | | | | | | |
| [Homo sapiens] | | | | | | |
| pyrin and HIN | gi\|41282051\|ref\|NP_945148.1\| | 50974.1 | LMSEMHSFIQIQK | 354 | 1623.8 | 379-391 |
| domain family, | | | | | | |
| member 1 beta 2 | | | | | | |
| isoform [Homo | | | | | | |
| sapiens] | | | | | | |
| transportin 1 | gi\|23510381\|ref\|NP_694858.1\| | 42709.1 | DVIVR | 355 | 601.37 | 299-303 |
| [Homo sapiens], | gi\|46409366\|ref\|NP_997255.1\| | | | | | |
| dedicator of | gi\|44889960\|ref\|NP_982272.1\| | 230806.7 | KEQTHWRQANEK | 356 | 1554.8 | 1332-1343 |
| cytokinesis 8 | | | | | | |
| [Homo sapiens] | | | | | | |
| kinesin family | gi\|9910266\|ref\|NP_064627.1\| | 160145.9 | QKETAKCEQQMAK | 357 | 1579.8 | 943-955 |
| member 15 [Homo | | | | | | |
| sapiens] | | | | | | |
| zinc finger protein | gi\|24429588\|ref\|NP_060651.2\| | 141677.7 | KEHGKQMK | 358 | 1001.5 | 1046-1053 |
| 532 [Homo | | | | | | |
| sapiens] | | | | | | |
| dehydrogenase E1 | gi\|38788380\|ref\|NP_061176.3\| | 103026.3 | IGGSVHLIVNNQLGYTTPAER | 359 | 2239.2 | 356-376 |
| and transketolase | | | | | | |
| domain containing | | | | | | |
| protein 1 [Homo | | | | | | |
| sapiens] | | | | | | |
| kinesin family | gi\|46852174\|ref\|NP_008985.3\| | 80025.3 | NKARINEDPK | 360 | 1184.6 | 347-356 |
| member 3A [Homo | | | | | | |
| sapiens] | | | | | | |
| major | gi\|4504415\|ref\|NP_002118.1\| | 38205.8 | THVTHHPVFDYEATLR | 361 | 1923 | 211-226 |
| histocompatibility | | | | | | |
| complex, class I, G | | | | | | |
| precursor [Homo | | | | | | |
| sapiens] | | | | | | |
| serine peptidase | gi\|74027261\|ref\|NP_006837.2\| | 120697.7 | ETCDEFRRLLQNGK | 362 | 1765.9 | 630-643 |
| inhibitor, Kazal | | | | | | |
| type 5 precursor | | | | | | |
| [Homo sapiens] | | | | | | |
| centaurin, gamma | gi\|16799069\|ref\|NP_114152.2\| | 95027 | VDSIGSGRAIPIK | 363 | 1312.8 | 358-370 |
| 3 [Homo sapiens] | | | | | | |
| PREDICTED: | gi\|113425692\|ref1XP_0011315 | 6923.8 | HGYIGDLKPLMITELR | 364 | 1872 | 44-59 |
| similarto | 13.1\| | | | | | |
| ribosomal protein | | | | | | |
| SiSa [Homo | | | | | | |
| sapiens] | | | | | | |
| RAB26, member | gi\|46361978\|ref\|NP_055168.2\| | 27882.5 | TPKSKGASTPAASTLPTANGA | 365 | 2084.1 | 6-27 |
| RAS oncogene | | | R | | | |
| family [Homo | | | | | | |
| sapiens] | | | | | | |
| PREDICTED: | gi\|89044999\|ref\|XP_934461.1\| | 28637.1 | LLEMQPKEAPEKDPEQLPK | 366 | 2220.2 | 96-114 |
| similar to Sorting | | | | | | |
| nexin-19 [Homo | | | | | | |
| sapiens] | | | | | | |
| hypothetical | gi\|40786410\|ref\|NP_955373.1\| | 64161 | GSQLEDQALR | 367 | 1116.6 | 529-538 |
| protein | | | | | | |
| LOC374920 | | | | | | |
| [Homo sapiens] | | | | | | |
| hypothetical | gi\|71067125\|ref\|NP_078857.4\| | 129514.6 | MDYSQEMHLKLSKK | 368 | 1753.9 | 1-14 |
| protein LOC79632 | | | | | | |
| [Homo sapiens] | | | | | | |
| fibronectin type III | gi\|34222186\|ref\|NP_660278.2\| | 38350.5 | AVSVNDEDLLVRILQGGR | 369 | 1954.1 | 117-134 |
| and ankyrin repeat | | | | | | |
| domains 1 [Homo | | | | | | |
| sapiens] | | | | | | |
| PREDICTED: | gi\|89035129\|ref\|XP_945446.1\| | 20879 | EEDEVFPQAQLEQSK | 370 | 1776.8 | 38-52 |
| similar to F52H3.5 | | | | | | |
| isoform 3 [Homo | | | | | | |
| sapiens] | | | | | | |
| CDC42-binding | gi\|16357474\|ref\|NP_006026.2\| | 194314 | IRPLNSEGTLNLLNCEPPR | 371 | 2193.1 | 1503-1521 |
| protein kinase beta | | | | | | |
| [Homo sapiens] | | | | | | |
| hect domain and | gi\|7705931\|ref\|NP_057407.1\| | 116834 | RTTEMMPVYLDLNK | 372 | 1726.9 | 434-447 |
| RLD S [Homo | | | | | | |
| sapiens] | | | | | | |
| hypothetical | gi\|32526911\|ref\|NP_057697.2\| | 57535.6 | QKCWQNGRVPK | 373 | 1400.7 | 341-351 |
| protein LOC51 313 | | | | | | |
| isoform 2 [Homo | | | | | | |
| sapiens] | | | | | | |
| neuraminidase | gi\|4557791\|ref\|NP_000425.1\| | 45449.3 | DGVFCLLSDDHGASWR | 374 | 1834.8 | 226-241 |
| precursor [Homo | | | | | | |
| sapiens] | | | | | | |
| upstream binding | gi\|7657671\|ref\|NP_055048.1\| | 89391.6 | KPAQEGGK | 375 | 814.44 | 394-401 |
| transcription | | | | | | |
| factor, RNA | | | | | | |
| polymerase I | | | | | | |
| [Homo sapiens] | | | | | | |
| sarcolemma | gi\|56550043\|ref\|NP_009090.2\| | 93187.1 | EKGNNK | 376 | 689.36 | 777-782 |
| associated protein | | | | | | |
| [Homo sapiens] | | | | | | |
| immunoglobulin- | gi\|28557777\|ref\|NP_787120.1\| | 57757 | FGHPCSMLSSLGSEWER | 377 | 2007.9 | 266-283 |
| like domain | | | | | | |
| containing | | | | | | |
| receptor 1 [Homo | | | | | | |
| sapiens] | | | | | | |
| Janus kinase 3 | gi\|47157315\|ref\|NP_000206.2\| | 125083.7 | EQGECLSLAVLDLARMAREQA | 378 | 2644.3 | 158-180 |
| [Homo sapiens] | | | QR | | | |
| regulatory solute | gi\|5730021\|ref\|NP_006502.1\| | 66771.3 | DLGQGIQNSVTDRPETR | 379 | 1885.9 | 523-539 |
| carrier protein, | | | | | | |
| family 1, member | | | | | | |
| 1 [Homo sapiens] | | | | | | |
| complement | gi\|4502511\|ref\|NP_001728.1\| | 63156.8 | AIEDYINEFSVR | 380 | 1455.7 | 497-508 |
| component 9 | | | | | | |
| [Homo sapiens] | | | | | | |
| PREDICTED: | gi\|113428147\|ref\|XP_038604. | 202106.8 | PTCELGDMSLLCVGVKK | 381 | 1922.9 | 82-98 |
| similar to Unc-13 | 10\| | | | | | |
| homolog A | | | | | | |
| (Muncl3-1) [Homo | | | | | | |
| sapiens] | | | | | | |
| apolipoprotein L1 | gi\|21735614\|ref\|NP_003652.21 | 43957.3 | ILQADQEL | 382 | 929.49 | 391-398 |
| isoform a | | | | | | |
| precursor [Homo | | | | | | |
| sapiens] | | | | | | |
| complement | gi\|4502503\|ref\|NP_000706.1\| | 67015 | LSLEIEQLELQR | 383 | 1470.8 | 574-585 |
| component 4 | | | | | | |
| binding protein, | | | | | | |
| alpha chain | | | | | | |
| precursor [Homo | | | | | | |
| sapiens] | | | | | | |
| cathelicidin | gi\|39753970\|ref\|NP_004336.2\| | 19284.3 | FALLGDFFR | 384 | 1085.6 | 132-140 |
| antimicrobial | | | | | | |
| peptide [Horno | | | | | | |
| sapiens] | | | | | | |
| ficolin 3 isoform 2 | gi\|27754778\|ref\|NP_775628.1\| | 31659.9 | YGIDWASGR | 385 | 1024.5 | 266-274 |
| precursor [Homo | | | | | | |
| sapiens] | | | | | | |
| sterile alpha motif | gi\|51339291\|ref\|NP_689916.2\| | 184522.8 | SNFDETYIENWRNILK | 386 | 2054.1 | 894-910 |
| domain containing | | | | | | |
| 9-like [Homo | | | | | | |
| sapiens] | | | | | | |
| OAF homolog | gi\|30425438\|ref\|NP_848602.1\| | 30670.4 | KPDGTLVSFTADFK | 387 | 1525.8 | 63-76 |
| [Homo sapiens] | | | | | | |
| gelsolin isoform b | gi\|38044288\|ref\|NP_937895.1\| | 80622.8 | EVQGFESATFLGYFK | 388 | 1722.8 | 97-111 |
| [Homo sapiens] | | | | | | |

### Example 3

### Cancer-Related Peptides Are Not Necessarily Biomarkers

The above methods showed that a number of peptides previously known to be assosciated with colorectal cancer were not indicative of a disease state, and, thus, not useful as a biomarker. Examples include, alpha-1-antitrypsin precursor (alpha-1 protease inhibitor) (alpha-1-antiproteinase), follistatin-related protein 5 precursor (follistatin-like 5), sodium-D-glucose cotransporter (regulatory solute carrier protein, family 1, member 1), hypothetical protein DKFZp781M0386, alpha-1-acid glycoprotein 1 precursor (AGP 1) (Orosomucoid-1) (OMD 1), complement component C9 precursor that Contains complement component C9a and complement component C9b, hypothetical protein, immunoglobulin J chain, serum amyloid A-4 protein precursor (constitutively expressed serum amyloid A protein) (C-SAA), apolipoprotein A-II precursor (Apo-AII) (ApoA-II) that contains apolipoprotein A-II (1-76), IGKC protein, serum albumin precursor, complement factor B precursor (EC 3.4.21.47) (C3/C5 convertase) (properdin factor B) (glycine-rich beta glycoprotein) (GBG) (PBF2) that contains complement factor B Ba fragment and complement factor B Bb fragment, hemopexin precursor (Beta-1B-glycoprotein), intercellular adhesion molecule 5 precursor (ICAM-5) (telencephalin), receptor interacting protein kinase 5, isoform 2, Ig heavy chain V-III region TIL, probable ATP-dependent RNA helicase DDX43 (EC 3.6.1.-) (DEAD-box protein 43) (DEAD-box protein HAGE) (helical antigen), FLJ10748 protein, hypothetical protein DKFZp686J11235 (fragment), C219-reactive peptide (FLJ39207), Ig kappa chain V-II region RPMI 6410 precursor, Ig kappa chain V-I region AU, homeobox protein Hox-A4 (Hox-1D) (Hox-1.4), cullin-4B (CUL-4B), zinc finger protein ZFPM1 (zinc finger protein multitype 1) (friend of GATA protein 1) (friend of GATA-1) (FOG-1), two-pore calcium channel protein 2 (two pore segment channel 2), stonin-2 (stoned B), hypothetical protein FLJ45653, hypothetical protein DKFZp434A128, Ig kappa chain V-I region CAR, ras-related protein Rap-1A (GTP-binding protein smg-p21A) (ras-related protein Krev-1) (C21KG) (G-22K), hypothetical protein FLJ37300, hypothetical protein FLJ36006, mirror-image polydactyly gene 1 protein, gamma-tubulin complex component 3 (GCP-3) (spindle pole body protein Spc98 homolog) (hSpc98) (hGCP3) (h104p), HERV-W_{—}7q21.2 provirus ancestral Env polyprotein precursor (envelope polyprotein) (HERV-7q Envelope protein) (HERV-W envelope protein) (syncytin) (syncytin 1) (enverin) (Env-W) that contains surface protein (SU) and transmembrane protein (TM), low-density lipoprotein receptor-related protein 2 precursor (megalin) (glycoprotein 330) (gp330), 40S ribosomal protein S16, Nuclear pore complex protein Nup214 (nucleoporin Nup214) (214 kDa nucleoporin) (CAN protein), cadherin EGF LAG seven-pass G-type receptor 1 precursor (flamingo homolog 2) (hFmi2), and KIAA0425 protein (fragment).

We claim:

1. A method for detecting colorectal cancer in a patient, comprising:
(i) obtaining a biological sample from said patient; and
(ii) evaluating said sample or a fraction of said sample for the presence of at least one biomarker selected from the group of peptides having the sequences of SEQ ID NOs: 176-388,
wherein the presence of said at least one biomarker is indicative of colorectal cancer.

2. The method according to claim 1, further comprising, prior to the evaluation step, harvesting low molecular weight peptides from said sample to generate at least one fraction comprising said peptides.

3. The method according to claim 1, wherein said biological sample is blood, serum or plasma.

4. The method according to claim 1, wherein the evaluation step comprises an assay selected from the group consisting of mass spectrometry, immunoassay, immuno-mass spectrometry.

5. The method according to claim 4, wherein said immunoassay is an enzyme linked immunosorbent assay or ELISA.

6. The method according to claim 4, wherein said mass spectrometry comprises multiple reaction monitoring (MRM).

7. The method according to claim 2, further comprising digesting said low molecular weight peptides.

8. The method according to claim 7, wherein said digestion comprises a trypsin digestion.

9. The method according to claim 1, wherein the colorectal cancer is in an early stage.

10. The method according to claim 1, wherein the colorectal cancer is in stage T1 or T2.

11. The method according to claim 1, wherein said evaluation step comprises evaluating said sample for the presence of at least biomarkers having the amino acid sequences of SEQ ID NOs: 176, 177 and 234.

12. A method for monitoring the progression of colorectal cancer in a patient, comprising:
(i) obtaining a biological sample from said patient;
(ii) evaluating said sample or a fraction of said sample for the presence of at least one biomarker selected from the group of peptides having the sequences of SEQ ID NOs: 176-388, wherein the presence of said at least one biomarker is indicative of colorectal cancer; and optionally
(iii) repeating steps (i) and (ii).

13. The method according to claim 12 further comprising, prior to the evaluation step, harvesting low molecular weight peptides from said sample to generate at least one fraction comprising said peptides.

14. The method according to claim 12, wherein said evaluation step comprises evaluating said sample for the presence of at least biomarkers having the amino acid sequences of SEQ ID NOs: 176, 177 and 234.

15. An antibody specific for a peptide selected from the group of peptides having the sequences of SEQ ID NOs: 176-388.

16. The antibody according to claim 15, wherein said antibody is a monoclonal antibody.

17. The antibody according to claim 15, wherein said antibody is a polyclonal antibody.

18. The antibody according to claim 15, wherein said antibody is a chimeric antibody.

19. The antibody according to claim 15, wherein the peptide is selected from the group of peptides having the sequences of SEQ ID NOs: 176, 177 and 234.

20. A kit for detecting colorectal cancer in a patient, comprising at least one antibody according to claim 15.


SEQUENCE LISTING (392 sequences)

SEQ ID NO: 1 - protein, 31 aa, Homo sapiens
TGPEAPRNGTVHLYLTKPLYTSAPSLQHLCR

SEQ ID NO: 2 - protein, 23 aa, Homo sapiens
VPSGLPDLKLLQVVYLHSNNITK

SEQ ID NO: 3 - protein, 17 aa, Homo sapiens
IVEKGYYSERDAADAVK

SEQ ID NO: 4 - protein, 12 aa, Homo sapiens
HHTGEKPYECEK

SEQ ID NO: 5 - protein, 15 aa, Homo sapiens
KQSVSVTLLILDISR

SEQ ID NO: 6 - protein, 20 aa, Homo sapiens
NYIILNMTENIDCEVFRQHR

SEQ ID NO: 7 - protein, 11 aa, Homo sapiens
YLTYDQLRKQK

SEQ ID NO: 8 - protein, 9 aa, Homo sapiens
SLLENAEAK

SEQ ID NO: 9 - protein, 16 aa, Homo sapiens
ENVMKLSIIQHDQPVK

SEQ ID NO: 10 - protein, 18 aa, Homo sapiens
TEILEGLNVNLTETPEAK

SEQ ID NO: 11 - protein, 17 aa, Homo sapiens
RQVEEAEEEIDRLESSK

SEQ ID NO: 12 - protein, 16 aa, Homo sapiens
RGKPEILELEGLILLK

SEQ ID NO: 13 - protein, 10 aa, Homo sapiens
INHEPTTFQK

SEQ ID NO: 14 - protein, 16 aa, Homo sapiens
TISHESGEHSAQGKTK

SEQ ID NO: 15 - protein, 30 aa, Homo sapiens
YVLYGNHRDSWVHGAVDPSSGTAVLLELSR

SEQ ID NO: 16 - protein, 17 aa, Homo sapiens
QLENEVKSTQEEIWTLR

SEQ ID NO: 17 - protein, 14 aa, Homo sapiens
EEIKHGQSLLALDK

SEQ ID NO: 18 - protein, 17 aa, Homo sapiens
ANPTQLNTVEFLWDPAK

SEQ ID NO: 19 - protein, 21 aa, Homo sapiens
DSPAHEVTGSGGAYMRFDVPR

SEQ ID NO: 20 - protein, 22 aa, Homo sapiens
NDPLLGMCSEMGFSGVRESFQR

SEQ ID NO: 21 - protein, 22 aa, Homo sapiens
SILEGTYSVPPHVSEPCHRLIR

SEQ ID NO: 22 - protein, 21 aa, Homo sapiens
YEYRVEMVHQSCNDPTKNIIR

SEQ ID NO: 23 - protein, 20 aa, Homo sapiens
VGLSGPPGAGKSTFIEYFGK

SEQ ID NO: 24 - protein, 10 aa, Homo sapiens
TFSLSSTLLR

SEQ ID NO: 25 - protein, 18 aa, Homo sapiens
FMMSKNPSVPVSAVFMDK

SEQ ID NO: 26 - protein, 17 aa, Homo sapiens
ETGFCGFGDSCKFLHDR

SEQ ID NO: 27 - protein, 12 aa, Homo sapiens
AAGQCNNMGQKR

SEQ ID NO: 28 - protein, 13 aa, Homo sapiens
ESITDIIKFMLQR

SEQ ID NO: 29 - protein, 11 aa, Homo sapiens
TSGPRPRGPLR

SEQ ID NO: 30 - protein, 22 aa, Homo sapiens
EREQQDLEFAKEMAEDDDDSFP

SEQ ID NO: 31 - protein, 14 aa, Homo sapiens
HGATVLTALGGILK

SEQ ID NO: 32 - protein, 13 aa, Homo sapiens
NVRSMPLIYWFSR

SEQ ID NO: 33 - protein, 16 aa, Homo sapiens
GVSLLLSVLRDPALQK

SEQ ID NO: 34 - protein, 11 aa, Homo sapiens
HYTRYYNGHTK

SEQ ID NO: 35 - protein, 14 aa, Homo sapiens
RGLRAVEVELEYQR

SEQ ID NO: 36 - protein, 22 aa, Homo sapiens
CLLCGALSELHVPPEWLAPGGK

SEQ ID NO: 37 - protein, 12 aa, Homo sapiens
RFLAVQSVISGR

SEQ ID NO: 38 - protein, 10 aa, Homo sapiens
ESDHSSLNNK

SEQ ID NO: 39 - protein, 14 aa, Homo sapiens
TPECVKGVVTDLLR

SEQ ID NO: 40 - protein, 10 aa, Homo sapiens
FQPTLLTLPR

SEQ ID NO: 41 - protein, 21 aa, Homo sapiens
PATQQAQKQSASTLQGEPRTK

SEQ ID NO: 42 - protein, 21 aa, Homo sapiens
ASHFQSHSSERQRHGSSQVWK

SEQ ID NO: 43 - protein, 13 aa, Homo sapiens
YENFEDPMGTIDK

SEQ ID NO: 44 - protein, 21 aa, Homo sapiens
LLEMLQSKGLSAKATEEDHER

SEQ ID NO: 45 - protein, 21 aa, Homo sapiens
AQETVPWNIILLLGGGFAMAK

SEQ ID NO: 46 - protein, 17 aa, Homo sapiens
NMLKEIDKTSFYSICNK

SEQ ID NO: 47 - protein, 16 aa, Homo sapiens
EEALLLMNKNIDVLEK

SEQ ID NO: 48 - protein, 16 aa, Homo sapiens
DMKPENLLCMGPELVK

SEQ ID NO: 49 - protein, 9 aa, Homo sapiens
NKTQISLVR

SEQ ID NO: 50 - protein, 15 aa, Homo sapiens
LRMLFKDDYPSSPPK

SEQ ID NO: 51 - protein, 15 aa, Homo sapiens
EYVDLEREAGKVYLK

SEQ ID NO: 52 - protein, 11 aa, Homo sapiens
DSERPSGIPER

SEQ ID NO: 53 - protein, 14 aa, Homo sapiens
YLAWYQQKPGQAPR

SEQ ID NO: 54 - protein, 8 aa, Homo sapiens
LTVLRQPK

SEQ ID NO: 55 - protein, 25 aa, Homo sapiens
PTEEEPGSPWSGPDELEPVVQDGQR

SEQ ID NO: 56 - protein, 12 aa, Homo sapiens
FNTLAKCPHCKK

SEQ ID NO: 57 - protein, 16 aa, Homo sapiens
LTEVADFHHAASKVLR

SEQ ID NO: 58 - protein, 7 aa, Homo sapiens
KAFVFPK

SEQ ID NO: 59 - protein, 13 aa, Homo sapiens
HGNTDSEGIVEVK

SEQ ID NO: 60 - protein, 26 aa, Homo sapiens
RIQNYSVMDICDYDESSGRWNCLVAR

SEQ ID NO: 61 - protein, 14 aa, Homo sapiens
SGVQQLIQYYQDQK

SEQ ID NO: 62 - protein, 10 aa, Homo sapiens
MIDVAEPGQR

SEQ ID NO: 63 - protein, 16 aa, Homo sapiens
LLIYYDDLLPSGVSDR

SEQ ID NO: 64 - protein, 12 aa, Homo sapiens
WAEESLHSLTSK

SEQ ID NO: 65 - protein, 15 aa, Homo sapiens
ELHVNHYRFSLSWPR

SEQ ID NO: 66 - protein, 29 aa, Homo sapiens
EAPFVLEASEIGSDLFAGVEAKGLSIRFK

SEQ ID NO: 67 - protein, 20 aa, Homo sapiens
LGLTMQYPEGYLEALANRER

SEQ ID NO: 68 - protein, 14 aa, Homo sapiens
KELEDCEVFRSTEK

SEQ ID NO: 69 - protein, 19 aa, Homo sapiens
KLLPLSVTPEAFSGTVIEK

SEQ ID NO: 70 - protein, 16 aa, Homo sapiens
LLIYGNSNRPSGVPDR

SEQ ID NO: 71 - protein, 17 aa, Homo sapiens
FDAEISQWKERGLGNLK

SEQ ID NO: 72 - protein, 16 aa, Homo sapiens
GTLPETPPLPLRSEMR

SEQ ID NO: 73 - protein, 7 aa, Homo sapiens
AELEKER

SEQ ID NO: 74 - protein, 17 aa, Homo sapiens
SAEHYTETALDEIRLLK

SEQ ID NO: 75 - protein, 24 aa, Homo sapiens
SHHPSESLLTVGSILNSLVRLQRS

SEQ ID NO: 76 - protein, 11 aa, Homo sapiens
VHEMEKEHLNK

SEQ ID NO: 77 - protein, 16 aa, Homo sapiens
AFRRGFHMVTNESPLK

SEQ ID NO: 78 - protein, 11 aa, Homo sapiens
ITCGGNNIGSK

SEQ ID NO: 79 - protein, 20 aa, Homo sapiens
APSCGFHHCHHGGLCLPSPK

SEQ ID NO: 80 - protein, 19 aa, Homo sapiens
AFSQNMHLIVHQRTHTGEK

SEQ ID NO: 81 - protein, 11 aa, Homo sapiens
DTERPSGIPER

SEQ ID NO: 82 - protein, 9 aa, Homo sapiens
IPTTFENGR

SEQ ID NO: 83 - protein, 16 aa, Homo sapiens
LLIYSNNQRPSGVPDR

SEQ ID NO: 84 - protein, 25 aa, Homo sapiens
ATQTESQPWIGSEGPSGPFVLSSPR

SEQ ID NO: 85 - protein, 16 aa, Homo sapiens
LLIYDNNKRPSGIPDR

SEQ ID NO: 86 - protein, 13 aa, Homo sapiens
SGDTASLTISGLR

SEQ ID NO: 87 - protein, 7 aa, Homo sapiens
VDPVNFK

SEQ ID NO: 88 - protein, 16 aa, Homo sapiens
LLLPPPAAWDLAVRLR

SEQ ID NO: 89 - protein, 19 aa, Homo sapiens
FLEERASSSLDSMPGPAGR

SEQ ID NO: 90 - protein, 18 aa, Homo sapiens
ELDIGISATYCGAHSVPK

SEQ ID NO: 91 - protein, 16 aa, Homo sapiens
LSGSNSGNTATLTISR

SEQ ID NO: 92 - protein, 18 aa, Homo sapiens
LSSATPTSLQVVWSTPAR

SEQ ID NO: 93 - protein, 21 aa, Homo sapiens
EMIKIPMPELERSQYPMEWGK

SEQ ID NO: 94 - protein, 12 aa, Homo sapiens
AFLDNAAEDLEK

SEQ ID NO: 95 - protein, 8 aa, Homo sapiens
ELESLVPR

SEQ ID NO: 96 - protein, 11 aa, Homo sapiens
YLNWYQQKPGK

SEQ ID NO: 97 - protein, 7 aa, Homo sapiens
NAECRGK

SEQ ID NO: 98 - protein, 24 aa, Homo sapiens
GTEPFVTGVRGQVPPLVTTNFLVK

SEQ ID NO: 99 - protein, 16 aa, Homo sapiens
LDKLVKLCIAMIDTGK

SEQ ID NO: 100 - protein, 15 aa, Homo sapiens
SYSCQVMHEGSTAEK

SEQ ID NO: 101 - protein, 10 aa, Homo sapiens
FSGSSSGADR

SEQ ID NO: 102 - protein, 15 aa, Homo sapiens
SSSTEPSLLVSWFTR

SEQ ID NO: 103 - protein, 10 aa, Homo sapiens
ITCSGDALPK

SEQ ID NO: 104 - protein, 7 aa, Homo sapiens
VFGGGTK

SEQ ID NO: 105 - protein, 23 aa, Homo sapiens
KGHIYQGSEADSVFSGFLIFPSA

SEQ ID NO: 106 - protein, 17 aa, Homo sapiens
IYWYQQKPGSPPQYLLR

SEQ ID NO: 107 - protein, 10 aa, Homo sapiens
ELCWLLRDER

SEQ ID NO: 108 - protein, 11 aa, Homo sapiens
DNERPSGIPER

SEQ ID NO: 109 - protein, 8 aa, Homo sapiens
RPSRSGGR

SEQ ID NO: 110 - protein, 18 aa, Homo sapiens
IVCLPSGIFFVCGTSAYR

SEQ ID NO: 111 - protein, 8 aa, Homo sapiens
EAVVAKPK

SEQ ID NO: 112 - protein, 9 aa, Homo sapiens
LLIYGTSSR

SEQ ID NO: 113 - protein, 16 aa, Homo sapiens
PKADSQLQPHGGDLTK

SEQ ID NO: 114 - protein, 24 aa, Homo sapiens
STLSIGRSLPHITDVSWRLEYQIK

SEQ ID NO: 115 - protein, 16 aa, Homo sapiens
LLIYDNNKRPSGVPDR

SEQ ID NO: 116 - protein, 16 aa, Homo sapiens
KLSQNIEILEDQIRAR

SEQ ID NO: 117 - protein, 10 aa, Homo sapiens
ITSASSQTLR

SEQ ID NO: 118 - protein, 14 aa, Homo sapiens
RQGQPIYNISSLLR

SEQ ID NO: 119 - protein, 24 aa, Homo sapiens
RTRTCTNPAPLNGGAFCEGQSVQK

SEQ ID NO: 120 - protein, 16 aa, Homo sapiens
IEFPPPNEEARLDILK

SEQ ID NO: 121 - protein, 23 aa, Homo sapiens
EPCALTLAQRNGQYELIIQLHEK

SEQ ID NO: 122 - protein, 12 aa, Homo sapiens
TRWTQNFHHFLK

SEQ ID NO: 123 - protein, 15 aa, Homo sapiens
AADISLDNLVEGKRK

SEQ ID NO: 124 - protein, 17 aa, Homo sapiens
KIDSLMNAVGCLKSEVK

SEQ ID NO: 125 - protein, 14 aa, Homo sapiens
NQAIRQPEVQAAPK

SEQ ID NO: 126 - protein, 20 aa, Homo sapiens
TEVEISDHNDSLLMKPLRFR

SEQ ID NO: 127 - protein, 15 aa, Homo sapiens
KMKATNSLGAGIIEK

SEQ ID NO: 128 - protein, 18 aa, Homo sapiens
DEDGSTLFRGWSQEGPVK

SEQ ID NO: 129 - protein, 17 aa, Homo sapiens
GDNAVIPYETDEDPRRR

SEQ ID NO: 130 - protein, 11 aa, Homo sapiens
RIATVLQHEER

SEQ ID NO: 131 - protein, 11 aa, Homo sapiens
RQAALIAYATR

SEQ ID NO: 132 - protein, 9 aa, Homo sapiens
PKNQAEAQK

SEQ ID NO: 133 - protein, 14 aa, Homo sapiens
LYNNFVNRNPLFEK

SEQ ID NO: 134 - protein, 7 aa, Homo sapiens
AHLTKLK

SEQ ID NO: 135 - protein, 22 aa, Homo sapiens
SLSPPQSQSKLSDSYSNTLPVR

SEQ ID NO: 136 - protein, 12 aa, Homo sapiens
QAVPDKVWKISK

SEQ ID NO: 137 - protein, 24 aa, Homo sapiens
QISVGFIGYPNVGKSSVINTLRSK

SEQ ID NO: 138 - protein, 17 aa, Homo sapiens
SFLFDKRPLSPALNALK

SEQ ID NO: 139 - protein, 19 aa, Homo sapiens
KFLALQSKNSDADFQNNEK

SEQ ID NO: 140 - protein, 21 aa, Homo sapiens
KMEHHFLATIGLFFVVAQGGK

SEQ ID NO: 141 - protein, 13 aa, Homo sapiens
VCSLERFRSLQDK

SEQ ID NO: 142 - protein, 23 aa, Homo sapiens
LANPHYQPELQAQATLINFTVTR

SEQ ID NO: 143 - protein, 14 aa, Homo sapiens
FYIFSQFMYDEHIK

SEQ ID NO: 144 - protein, 16 aa, Homo sapiens
TGLFTPDMAFEAIVKK

SEQ ID NO: 145 - protein, 28 aa, Homo sapiens
ILVFGGMVEYGRYSNELYELQASRWLWK

SEQ ID NO: 146 - protein, 10 aa, Homo sapiens
HFNIHSWEKK

SEQ ID NO: 147 - protein, 15 aa, Homo sapiens
KRVLNVTNLEFSDTR

SEQ ID NO: 148 - protein, 23 aa, Homo sapiens
AVGDGIVLCKMINLSVPDTIDER

SEQ ID NO: 149 - protein, 13 aa, Homo sapiens
GLYDDTTAFLDEK

SEQ ID NO: 150 - protein, 14 aa, Homo sapiens
RLAEINREFLCDQK

SEQ ID NO: 151 - protein, 26 aa, Homo sapiens
ANCVAWLGGAIFGALQDILGSRSVSK

SEQ ID NO: 152 - protein, 16 aa, Homo sapiens
FHPSMLSNYLKSLKVK

SEQ ID NO: 153 - protein, 10 aa, Homo sapiens
NGVSEENLAK

SEQ ID NO: 154 - protein, 21 aa, Homo sapiens
QVLELVSSDSLNVPSEEEVYR

SEQ ID NO: 155 - protein, 24 aa, Homo sapiens
YEPLGLDAAGIEEAITAVGSFILK

SEQ ID NO: 156 - protein, 12 aa, Homo sapiens
IQYTVYDRAENK

SEQ ID NO: 157 - protein, 15 aa, Homo sapiens
AHTGEKSCQCNECGK

SEQ ID NO: 158 - protein, 31 aa, Homo sapiens
RVGHAGFELLTSGDLPASASQSAGIAGVSHR

SEQ ID NO: 159 - protein, 22 aa, Homo sapiens
SRSSHARPVSLATSGGSEEEDK

SEQ ID NO: 160 - protein, 16 aa, Homo sapiens
KMHEGDLAMALVLDEK

SEQ ID NO: 161 - protein, 14 aa, Homo sapiens
VTNSSIVVSPKMTR

SEQ ID NO: 162 - protein, 21 aa, Homo sapiens
EMGKDYFDYINVGSWDNGELK

SEQ ID NO: 163 - protein, 27 aa, Homo sapiens
NINLPEIPRNINIGDLNIKVPNLSQFV

SEQ ID NO: 164 - protein, 14 aa, Homo sapiens
VYRAEYINFLENLK

SEQ ID NO: 165 - protein, 28 aa, Homo sapiens
NWEMAYTDTGMIYFIDHNTKTTTWLDPR

SEQ ID NO: 166 - protein, 22 aa, Homo sapiens
KNTIMFDDIGRNFLMNPQNGLK

SEQ ID NO: 167 - protein, 23 aa, Homo sapiens
NGVFRVQKFGHGSVEYLALNIDK

SEQ ID NO: 168 - protein, 19 aa, Homo sapiens
DQTKCAASVGIGSNILLAR

SEQ ID NO: 169 - protein, 19 aa, Homo sapiens
KNGGEWDVEKVVIDLGGSR

SEQ ID NO: 170 - protein, 13 aa, Homo sapiens
SPFNSPSPQDSPR

SEQ ID NO: 171 - protein, 17 aa, Homo sapiens
DKLRPIIISMNYSLPLR

SEQ ID NO: 172 - protein, 31 aa, Homo sapiens
VIAAMTVGKDVSSLFPDVVNCMQTDNLELKK

SEQ ID NO: 173 - protein, 14 aa, Homo sapiens
VLLAFRGRGSGTDR

SEQ ID NO: 174 - protein, 16 aa, Homo sapiens
DASVPLIDVTNLPTPR

SEQ ID NO: 175 - protein, 16 aa, Homo sapiens
AKQLAALTLEDEEEVK

SEQ ID NO: 176 - protein, 7 aa, Homo sapiens
KAFVFPK

SEQ ID NO: 177 - protein, 12 aa, Homo sapiens
RQDNEILIFWSK

SEQ ID NO: 178 - protein, 18 aa, Homo sapiens
ASQSISSWLAWYQQKPGK

SEQ ID NO: 179 - protein, 9 aa, Homo sapiens
IPTTFENGR

SEQ ID NO: 180 - protein, 22 aa, Homo sapiens
SWVPHTFESELSDPVELLVAES

SEQ ID NO: 181 - protein, 22 aa, Homo sapiens
TPGAAANLELIFVGPQHAGNYR

SEQ ID NO: 182 - protein, 8 aa, Homo sapiens
RPSGVSDR

SEQ ID NO: 183 - protein, 19 aa, Homo sapiens
SYELTQPPSVSVSPGQTAR

SEQ ID NO: 184 - protein, 13 aa, Homo sapiens
EPFERLVSAFRDK

SEQ ID NO: 185 - protein, 11 aa, Homo sapiens
NDQRPSGVPDR

SEQ ID NO: 186 - protein, 8 aa, Homo sapiens
LTVLGQPK

SEQ ID NO: 187 - protein, 14 aa, Homo sapiens
YESEIKYLTIMKLK

SEQ ID NO: 188 - protein, 8 aa, Homo sapiens
LTVLRQPK

SEQ ID NO: 189 - protein, 16 aa, Homo sapiens
YHQPQRASGSSYGGVK

SEQ ID NO: 190 - protein, 14 aa, Homo sapiens
SFFSFLGEAFDGAR

SEQ ID NO: 191 - protein, 10 aa, Homo sapiens
ANRPFLVFIR

SEQ ID NO: 192 - protein, 12 aa, Homo sapiens
AYSLFSYNTQGR

SEQ ID NO: 193 - protein, 15 aa, Homo sapiens
YAASSYLSLTPEQWR

SEQ ID NO: 194 - protein, 13 aa, Homo sapiens
VNVEDAGGETLGR

SEQ ID NO: 195 - protein, 12 aa, Homo sapiens
QIKDETLQAAVR

SEQ ID NO: 196 - protein, 18 aa, Homo sapiens
QFTKQNGETPGIIAEASK

SEQ ID NO: 197 - protein, 12 aa, Homo sapiens
AATGECTATVGK

SEQ ID NO: 198 - protein, 11 aa, Homo sapiens
TVGSDTFYSFK

SEQ ID NO: 199 - protein, 15 aa, Homo sapiens
YNSQNQSNNQFVLYR

SEQ ID NO: 200 - protein, 13 aa, Homo sapiens
FSGSGSGTDFTLK

SEQ ID NO: 201 - protein, 16 aa, Homo sapiens
FSGSGSGTDFTLKISR

SEQ ID NO: 202 - protein, 10 aa, Homo sapiens
VYEMYVTVHR

SEQ ID NO: 203 - protein, 10 aa, Homo sapiens
FSGSSSGADR

SEQ ID NO: 204 - protein, 16 aa, Homo sapiens
WGMLFLVNQLFKIYIK

SEQ ID NO: 205 - protein, 9 aa, Homo sapiens
VTVSSASTK

SEQ ID NO: 206 - protein, 13 aa, Homo sapiens
SGDTASLTISGLR

SEQ ID NO: 207 - protein, 18 aa, Homo sapiens
RSPKPCLAQPAQAPGTLR

SEQ ID NO: 208 - protein, 20 aa, Homo sapiens
FWGKSVEAEPRGTITLELLK

SEQ ID NO: 209 - protein, 7 aa, Homo sapiens
AFLLTPR

SEQ ID NO: 210 - protein, 14 aa, Homo sapiens
KWIYHLTEGSTDLR

SEQ ID NO: 211 - protein, 16 aa, Homo sapiens
LLIYSNNQRPSGVPDR

SEQ ID NO: 212 - protein, 7 aa, Homo sapiens
TFGQGTK

SEQ ID NO: 213 - protein, 14 aa, Homo sapiens
YLAWYQQKPGQAPR

SEQ ID NO: 214 - protein, 14 aa, Homo sapiens
SGVQQLIQYYQDQK

SEQ ID NO: 215 - protein, 25 aa, Homo sapiens
AAVSSSSTAAPAGPAKSVLKSAEGK

SEQ ID NO: 216 - protein, 11 aa, Homo sapiens
ITCGGNNIGSK

SEQ ID NO: 217 - protein, 13 aa, Homo sapiens
ETGEMFVNDRLDR

SEQ ID NO: 218 - protein, 19 aa, Homo sapiens
KNGGEWDVEKVVIDLGGSR

SEQ ID NO: 219 - protein, 11 aa, Homo sapiens
DSERPSGIPER

SEQ ID NO: 220 - protein, 22 aa, Homo sapiens
ALIAEHQTFMEEMTRKQPDVDR

SEQ ID NO: 221 - protein, 26 aa, Homo sapiens
ALRLLLLVQLVAGRCGLAGAAGGAQR

SEQ ID NO: 222 - protein, 18 aa, Homo sapiens
HKVTGVEYHNAGTQTVPK

SEQ ID NO: 223 - protein, 20 aa, Homo sapiens
PEVAAKPALPTQKPAGTLPR

SEQ ID NO: 224 - protein, 17 aa, Homo sapiens
IYWYQQKPGSPPQYLLR

SEQ ID NO: 225 - protein, 15 aa, Homo sapiens
KRVLNVTNLEFSDTR

SEQ ID NO: 226 - protein, 16 aa, Homo sapiens
HIDPFDLTHFLAQGRR

SEQ ID NO: 227 - protein, 23 aa, Homo sapiens
TRNQTQHLAPQPVLLSDMLYCLK

SEQ ID NO: 228 - protein, 21 aa, Homo sapiens
PGPCWPGPSSHANGDPVAVAK

SEQ ID NO: 229 - protein, 17 aa, Homo sapiens
AVIELLEKSGVNLDGKK

SEQ ID NO: 230 - protein, 13 aa, Homo sapiens
TMLPEATLLIMIR

SEQ ID NO: 231 - protein, 11 aa, Homo sapiens
MAVTGAGSGAR

SEQ ID NO: 232 - protein, 9 aa, Homo sapiens
SLLENAEAK

SEQ ID NO: 233 - protein, 22 aa, Homo sapiens
QKTLVEQLLSLLNSSPGPPTRK

SEQ ID NO: 234 - protein, 16 aa, Homo sapiens
LSGSNSGNTATLTISR

SEQ ID NO: 235 - protein, 29 aa, Homo sapiens
ITSVFSLQSQQASEFLPPEVNQLLQDVLK

SEQ ID NO: 236 - protein, 24 aa, Homo sapiens
VISFIENTSTPVDRHVSSSDRVGK

SEQ ID NO: 237 - protein, 8 aa, Homo sapiens
ELESLVPR

SEQ ID NO: 238 - protein, 11 aa, Homo sapiens
LVSPEQPPKAK

SEQ ID NO: 239 - protein, 10 aa, Homo sapiens
ITCSGDALPK

SEQ ID NO: 240 - protein, 8 aa, Homo sapiens
QAEVHTGR

SEQ ID NO: 241 - protein, 13 aa, Homo sapiens
HGNTDSEGIVEVK

SEQ ID NO: 242 - protein, 16 aa, Homo sapiens
LLIYYDDLLPSGVSDR

SEQ ID NO: 243 - protein, 16 aa, Homo sapiens
NNMPCSAFSAKSPSPR

SEQ ID NO: 244 - protein, 19 aa, Homo sapiens
KLLPLSVTPEAFSGTVIEK

SEQ ID NO: 245 - protein, 24 aa, Homo sapiens
LYAVHQEGNKNVGLDIEAEVPAVK

SEQ ID NO: 246 - protein, 24 aa, Homo sapiens
EDTAAAVMYTVVTPLLNPFIYSMR

SEQ ID NO: 247 - protein, 12 aa, Homo sapiens
STDYGIFQINSR

SEQ ID NO: 248 - protein, 18 aa, Homo sapiens
RSLNNLQDLAHNPGLSPR

SEQ ID NO: 249 - protein, 18 aa, Homo sapiens
EKDPQPQQLPPMDPKLLK

SEQ ID NO: 250 - protein, 20 aa, Homo sapiens
FFGHGAEDSLADQAANEWGR

SEQ ID NO: 251 - protein, 15 aa, Homo sapiens
GPGGAWAAEVISDAR

SEQ ID NO: 252 - protein, 16 aa, Homo sapiens
RGPGGAWAAEVISDAR

SEQ ID NO: 253 - protein, 14 aa, Homo sapiens
SFFSFLGEAFDGAR

SEQ ID NO: 254 - protein, 10 aa, Homo sapiens
LLVVYPWTQR

SEQ ID NO: 255 - protein, 12 aa, Homo sapiens
VVAGVANALAHK

SEQ ID NO: 256 - protein, 23 aa, Homo sapiens
VYLQGLIDYYLFGNSSTVLEDSK

SEQ ID NO: 257 - protein, 16 aa, Homo sapiens
DALSSVQESQVAQQAR

SEQ ID NO: 258 - protein, 11 aa, Homo sapiens
GWVTDGFSSLK

SEQ ID NO: 259 - protein, 15 aa, Homo sapiens
AGPHCPTAQLIATLK

SEQ ID NO: 260 - protein, 9 aa, Homo sapiens
HITSLEVIK

SEQ ID NO: 261 - protein, 11 aa, Homo sapiens
ICLDLQAPLYK

SEQ ID NO: 262 - protein, 12 aa, Homo sapiens
KICLDLQAPLYK

SEQ ID NO: 263 - protein, 7 aa, Homo sapiens
AWFLESK

SEQ ID NO: 264 - protein, 13 aa, Homo sapiens
DGWQWFWSPSTFR

SEQ ID NO: 265 - protein, 9 aa, Homo sapiens
ELLETVVNR

SEQ ID NO: 266 - protein, 12 aa, Homo sapiens
GFMQTYYDDHLR

SEQ ID NO: 267 - protein, 11 aa, Homo sapiens
DQYYNIDVPSR

SEQ ID NO: 268 - protein, 16 aa, Homo sapiens
GLPNVVTSAISLPNIR

SEQ ID NO: 269 - protein, 12 aa, Homo sapiens
SWFEPLVEDMQR

SEQ ID NO: 270 - protein, 20 aa, Homo sapiens
HTFMGVVSLGSPSGEVSHPR

SEQ ID NO: 271 - protein, 19 aa, Homo sapiens
AVLDVFEEGTEASAATAVK

SEQ ID NO: 272 - protein, 22 aa, Homo sapiens
FNRPFLMIIVPTDTQNIFFMSK

SEQ ID NO: 273 - protein, 11 aa, Homo sapiens
ITLLSALVETR

SEQ ID NO: 274 - protein, 18 aa, Homo sapiens
FNAVLTNPQGDYDTSTGK

SEQ ID NO: 275 - protein, 12 aa, Homo sapiens
ACIPTGPYPCGK

SEQ ID NO: 276 - protein, 7 aa, Homo sapiens
NCELFTR

SEQ ID NO: 277 - protein, 13 aa, Homo sapiens
AEMADQAAAWLTR

SEQ ID NO: 278 - protein, 7 aa, Homo sapiens
DTIFPSR

SEQ ID NO: 279 - protein, 12 aa, Homo sapiens
ALQDQLVLVAAK

SEQ ID NO: 280 - protein, 11 aa, Homo sapiens
EIMENYNIALR

SEQ ID NO: 281 - protein, 13 aa, Homo sapiens
FNTFIHEDIWNIR

SEQ ID NO: 282 - protein, 8 aa, Homo sapiens
YCNLMMQR

SEQ ID NO: 283 - protein, 12 aa, Homo sapiens
DTDTGALLFIGK

SEQ ID NO: 284 - protein, 7 aa, Homo sapiens
AFQYHSK

SEQ ID NO: 285 - protein, 7 aa, Homo sapiens
KSSIIIR

SEQ ID NO: 286 - protein, 8 aa, Homo sapiens
PGIVFITK

SEQ ID NO: 287 - protein, 15 aa, Homo sapiens
FYNNHAFEEQEPPEK

SEQ ID NO: 288 - protein, 12 aa, Homo sapiens
FRKQIQESEHMK

SEQ ID NO: 289 - protein, 15 aa, Homo sapiens
EPVYLSGYGVELAIK

SEQ ID NO: 290 - protein, 6 aa, Homo sapiens
EPANSR

SEQ ID NO: 291 - protein, 15 aa, Homo sapiens
FNKPFVFLMIEQNTK

SEQ ID NO: 292 - protein, 22 aa, Homo sapiens
GTEAAGAMFLEAIPMSIPPEVK

SEQ ID NO: 293 - protein, 10 aa, Homo sapiens
LSITGTYDLK

SEQ ID NO: 294 - protein, 7 aa, Homo sapiens
SPLFMGK

SEQ ID NO: 295 - protein, 10 aa, Homo sapiens
SVLGQLGITK

SEQ ID NO: 296 - protein, 18 aa, Homo sapiens
VFSNGADLSGVTEEAPLK

SEQ ID NO: 297 - protein, 19 aa, Homo sapiens
LLLSSLGIPVNHLIEGSQK

SEQ ID NO: 298 - protein, 19 aa, Homo sapiens
TDQEIKILEENKELENALK

SEQ ID NO: 299 - protein, 12 aa, Homo sapiens
WNHCFLCKKCVK

SEQ ID NO: 300 - protein, 11 aa, Homo sapiens
LAEGFPLPLLK

SEQ ID NO: 301 - protein, 14 aa, Homo sapiens
IALGGLLFPASNLR

SEQ ID NO: 302 - protein, 15 aa, Homo sapiens
AEAEEDGDLQCLCVK

SEQ ID NO: 303 - protein, 16 aa, Homo sapiens
AEAFFPNMVNMLVLGK

SEQ ID NO: 304 - protein, 11 aa, Homo sapiens
SLESSQRQEAR

SEQ ID NO: 305 - protein, 14 aa, Homo sapiens
GSCFIQTLVDVFTK

SEQ ID NO: 306 - protein, 13 aa, Homo sapiens
LGNQEPGGQTALK

SEQ ID NO: 307 - protein, 10 aa, Homo sapiens
FYTIEILKVE

SEQ ID NO: 308 - protein, 12 aa, Homo sapiens
ACEGAEVREAAR

SEQ ID NO: 309 - protein, 15 aa, Homo sapiens
LPEMEPLVPRVKEVK

SEQ ID NO: 310 - protein, 17 aa, Homo sapiens
SASEGSSKKSGSGNSGK

SEQ ID NO: 311 - protein, 13 aa, Homo sapiens
RYRVYPEGTLELR

SEQ ID NO: 312 - protein, 17 aa, Homo sapiens
CSELKGQLGEAEGENLR

SEQ ID NO: 313 - protein, 22 aa, Homo sapiens
CVRADEYSLYAKNENTGVVVVK

SEQ ID NO: 314 - protein, 18 aa, Homo sapiens
HMKIKSLEEMYVFSLPMK

SEQ ID NO: 315 - protein, 14 aa, Homo sapiens
FIVDYSETSPQCPK

SEQ ID NO: 316 - protein, 14 aa, Homo sapiens
ILNTPNCALQIVAR

SEQ ID NO: 317 - protein, 9 aa, Homo sapiens
TDFLIFDPK

SEQ ID NO: 318 - protein, 17 aa, Homo sapiens
PSSSVSLLCRPVCRPAR

SEQ ID NO: 319 - protein, 11 aa, Homo sapiens
KVQPVDLDKAR

SEQ ID NO: 320 - protein, 13 aa, Homo sapiens
ENSRTETTMSPPR

SEQ ID NO: 321 - protein, 12 aa, Homo sapiens
SHSAHFFEFLTK

SEQ ID NO: 322 - protein, 23 aa, Homo sapiens
AITPPHPASQANIIFDITEGNLR

SEQ ID NO: 323 - protein, 9 aa, Homo sapiens
LLCGLLAER

SEQ ID NO: 324 - protein, 13 aa, Homo sapiens
TAFQEALDAAGDK

SEQ ID NO: 325 - protein, 15 aa, Homo sapiens
QCGLHDGLQDTSQDK

SEQ ID NO: 326 - protein, 19 aa, Homo sapiens
SALYSPSDPLTLLQADTVR

SEQ ID NO: 327 - protein, 10 aa, Homo sapiens
VHFEDLVFLR

SEQ ID NO: 328 - protein, 11 aa, Homo sapiens
SVIMVVFAEDK

SEQ ID NO: 329 - protein, 13 aa, Homo sapiens
EENLLNVPKPLPK

SEQ ID NO: 330 - protein, 11 aa, Homo sapiens
KVLTSLGDAIK

SEQ ID NO: 331 - protein, 13 aa, Homo sapiens
EISNLNQLIEEFK

SEQ ID NO: 332 - protein, 20 aa, Homo sapiens
MNQLILTTGGRINTFHGTKK

SEQ ID NO: 333 - protein, 12 aa, Homo sapiens
FQDNLDFIQWFK

SEQ ID NO: 334 - protein, 16 aa, Homo sapiens
HLGLYESSMMPPGRPR

SEQ ID NO: 335 - protein, 13 aa, Homo sapiens
ASASDGSSFVVAR

SEQ ID NO: 336 - protein, 11 aa, Homo sapiens
SNSSMHITDCR

SEQ ID NO: 337 - protein, 10 aa, Homo sapiens
DAVLLVFANK

SEQ ID NO: 338 - protein, 11 aa, Homo sapiens
SFQTGLFTAAR

SEQ ID NO: 339 - protein, 10 aa, Homo sapiens
TYHVGEQWQK

SEQ ID NO: 340 - protein, 14 aa, Homo sapiens
HGATVLTALGGILK

SEQ ID NO: 341 - protein, 10 aa, Homo sapiens
LGDNEETQVR

SEQ ID NO: 342 - protein, 10 aa, Homo sapiens
PGLLHQGRQR

SEQ ID NO: 343 - protein, 9 aa, Homo sapiens
YEQLQNETR

SEQ ID NO: 344 - protein, 11 aa, Homo sapiens
QSVEADINGLR

SEQ ID NO: 345 - protein, 10 aa, Homo sapiens
FRMTQLTVEK

SEQ ID NO: 346 - protein, 10 aa, Homo sapiens
EGEMEVAMQK

SEQ ID NO: 347 - protein, 19 aa, Homo sapiens
MAASVVCRAATAGAQVLLR

SEQ ID NO: 348 - protein, 10 aa, Homo sapiens
QNLLDDLVTR

SEQ ID NO: 349 - protein, 15 aa, Homo sapiens
IAADMSNIIENLDTR

SEQ ID NO: 350 - protein, 15 aa, Homo sapiens
LSWKMPDDDGGDRIK

SEQ ID NO: 351 - protein, 15 aa, Homo sapiens
TAELSVKEYKEVNEK

SEQ ID NO: 352 - protein, 8 aa, Homo sapiens
DSREEVER

SEQ ID NO: 353 - protein, 21 aa, Homo sapiens
SQSADPFLNLEMDAGISNIQR

SEQ ID NO: 354 - protein, 13 aa, Homo sapiens
LMSEMHSFIQIQK

SEQ ID NO: 355 - protein, 5 aa, Homo sapiens
DVIVR

SEQ ID NO: 356 - protein, 12 aa, Homo sapiens
KEQTHWRQANEK

SEQ ID NO: 357 - protein, 13 aa, Homo sapiens
QKETAKCEQQMAK

SEQ ID NO: 358 - protein, 8 aa, Homo sapiens
KEHGKQMK

SEQ ID NO: 359 - protein, 21 aa, Homo sapiens
IGGSVHLIVNNQLGYTTPAER

SEQ ID NO: 360 - protein, 10 aa, Homo sapiens
NKARINEDPK

SEQ ID NO: 361 - protein, 16 aa, Homo sapiens
THVTHHPVFDYEATLR

SEQ ID NO: 362 - protein, 14 aa, Homo sapiens
ETCDEFRRLLQNGK

SEQ ID NO: 363 - protein, 13 aa, Homo sapiens
VDSIGSGRAIPIK

SEQ ID NO: 364 - protein, 16 aa, Homo sapiens
HGYIGDLKPLMITELR

SEQ ID NO: 365 - protein, 22 aa, Homo sapiens
TPKSKGASTPAASTLPTANGAR

SEQ ID NO: 366 - protein, 19 aa, Homo sapiens
LLEMQPKEAPEKDPEQLPK

SEQ ID NO: 367 - protein, 10 aa, Homo sapiens
GSQLEDQALR

SEQ ID NO: 368 - protein, 14 aa, Homo sapiens
MDYSQEMHLKLSKK

SEQ ID NO: 369 - protein, 18 aa, Homo sapiens
AVSVNDEDLLVRILQGGR

SEQ ID NO: 370 - protein, 15 aa, Homo sapiens
EEDEVFPQAQLEQSK

SEQ ID NO: 371 - protein, 19 aa, Homo sapiens
IRPLNSEGTLNLLNCEPPR

SEQ ID NO: 372 - protein, 14 aa, Homo sapiens
RTTEMMPVYLDLNK

SEQ ID NO: 373 - protein, 11 aa, Homo sapiens
QKCWQNGRVPK

SEQ ID NO: 374 - protein, 16 aa, Homo sapiens
DGVFCLLSDDHGASWR

SEQ ID NO: 375 - protein, 8 aa, Homo sapiens
KPAQEGGK

SEQ ID NO: 376 - protein, 6 aa, Homo sapiens
EKGNNK

SEQ ID NO: 377 - protein, 18 aa, Homo sapiens
FGHPCSMLSSLGSEVVER

SEQ ID NO: 378 - protein, 23 aa, Homo sapiens
EQGECLSLAVLDLARMAREQAQR

SEQ ID NO: 379 - protein, 17 aa, Homo sapiens
DLGQGIQNSVTDRPETR

SEQ ID NO: 380 - protein, 12 aa, Homo sapiens
AIEDYINEFSVR

SEQ ID NO: 381 - protein, 17 aa, Homo sapiens
PTCELGDMSLLCVGVKK

SEQ ID NO: 382 - protein, 8 aa, Homo sapiens
ILQADQEL

SEQ ID NO: 383 - protein, 12 aa, Homo sapiens
LSLEIEQLELQR

SEQ ID NO: 384 - protein, 9 aa, Homo sapiens
FALLGDFFR

SEQ ID NO: 385 - protein, 9 aa, Homo sapiens
YGIDWASGR

SEQ ID NO: 386 - protein, 17 aa, Homo sapiens
SNFDETYIENVVRNILK

SEQ ID NO: 387 - protein, 14 aa, Homo sapiens
KPDGTLVSFTADFK

SEQ ID NO: 388 - protein, 15 aa, Homo sapiens
EVQGFESATFLGYFK

SEQ ID NO: 389 - protein, 12 aa, Homo sapiens
RQDNEILIFWSK

SEQ ID NO: 390 - protein, 15 aa, Homo sapiens
YEVQGEVFTKPQLWP

SEQ ID NO: 391 - protein, 7 aa, Homo sapiens
VLNQELR

SEQ ID NO: 392 - protein, 4 aa, Artificial Sequence
  Description of Artificial Sequence Synthetic peptide
DEAD